(19) United States
(12) Patent Application Publication
(10) Pub. No.: US 2014/0228248 A1
(43) Pub. Date: Aug. 14, 2014

(54) METHODS AND COMPOSITIONS FOR DETECTING INFECTIONS

(71) Applicant: BIOMED VALLEY DISCOVERIES, INC., Kansas City, MO (US)

(72) Inventors: Saurabh Saha, Leawood, KS (US); Chetan Bettegowda, Perry Hall, MD (US); David Tung, Lee's Summit, MO (US)

(73) Assignee: BIOMED VALLEY DISCOVERIES, INC., Kansas City, KS (US)

(21) Appl. No.: 14/347,463

(22) PCT Filed: Sep. 24, 2012
(86) PCT No.: PCT/US2012/056865

§ 371 Date: Mar. 26, 2014

Related U.S. Application Data
(60) Provisional application No. 61/579,020, filed on Dec. 22, 2011.
(60) Provisional application No. 61/540,158, filed on Sep. 28, 2011.

(51) Int. Cl. C12Q 1/18; C12Q 1/16; C12Q 1/48; C12Q 1/04

(52) U.S. Cl. CPC C12Q 1/18; C12Q 1/04; C12Q 1/16; C12Q 1/485
USPC 506/10; 435/34; 435/5; 435/35; 435/15; 435/287.1; 435/32; 506/15

(57) ABSTRACT

The present invention provides, inter alia, methods for detecting whether a subject has an infection. These methods include (a) incubating a test sample from a subject suspected of having an infection with a labeled molecule, such as a labeled nucleoside analog, that is preferentially incorporated into a pathogenic microorganism for a period of time sufficient for the pathogenic microorganism to incorporate the labeled molecule; (b) removing any unincorporated labeled molecule from the test sample; and (c) detecting the labeled molecule within the pathogenic microorganism, if any, in the test sample, wherein the presence of labeled molecule within the pathogenic microorganism indicates that the subject has an infection.

### CROSS-REFERENCE TO RELATED APPLICATIONS

The present invention claims benefit to U.S. provisional application Ser. Nos. 61/579,020 filed Dec. 22, 2011 and 61/540,158 filed Sep. 28, 2011. The entire contents of the above applications are incorporated by reference.

### FIELD OF THE INVENTION

The present invention provides, inter alia, methods and compositions for detecting infections using labeled molecules that are preferentially incorporated into a pathogenic microorganism.

### BACKGROUND OF THE INVENTION

Despite affecting millions of people annually and being one of the leading causes of death worldwide, bacterial pneumonia remains a challenge to diagnose. Acute respiratory infections are the number one cause of death worldwide and bacterial pneumonia is responsible for a large fraction of those deaths. Though healthcare costs attributable to pneumonia are in excess of billions of dollars annually, its definitive diagnosis often remains elusive. The sensitivity (47-69%) and specificity (58-75%) of the clinical examination alone are extremely low, making adjunct tests exceptionally important in the decision making process. Most definitions of pneumonia require the presence of an infiltrate on chest x-ray, but even that finding is non-specific, as infiltrates can be caused by multiple etiologies. Additionally, early in the infection process infiltrates may not be visible on x-ray. Therefore, many clinicians order large numbers of additional tests including computed tomography (CT) scans of the chest, complete blood counts, arterial blood gas, immune serologies and more recently, expensive polymerase chain reaction (PCR) based assays to aid in the diagnosis. Despite these and other tools, the formal diagnosis of pneumonia is still widely variable and largely rests on the judgment of the individual clinician treating the patient.

Accurate and early diagnosis is critical for the successful treatment of pneumonia and limiting morbidity and mortality. Central to initiating appropriate therapy is the detection of the causative agent. Pneumonia can be caused by a number of microbes but is most commonly attributed to viral or bacterial etiologies. While the treatment of viral pneumonia is mostly supportive, the treatment of bacterial pneumonia entails rapid initiation of broad spectrum antibiotics, especially in those patients with chronic illnesses, underlying pulmonary dysfunction, immuno-compromised states, cancer, or who are at extremes of age. Current strategies to determine the etiology include invasive bronchoscopic or tracheal sputum sampling and needle aspiration of pleural effusions. The most routine, inexpensive and non-invasive approach is the use of gram stain and sputum culture. However, the ability to identify whether a patient has bacterial pneumonia using these two techniques is inadequate, with reported sensitivities as low as 50%.

The sensitivity declines even further when attempting to detect atypical organisms such as Mycoplasma pneumoniae and Legionella pneumophila. Atypical organisms account for up to 40-50% of all community acquired bacterial pneumonia cases but are not seen on gram stain and are exceedingly difficult to culture. When provided with negative gram stain and culture results, many clinicians do not initiate antibiotics or stop them after an abbreviated course. As a result, atypical organisms are a leading cause of treatment failures in patients with pneumonia.

In addition to contributing to treatment failures, the inability to accurately detect whether a patient has bacterial pneumonia has led to the misuse of antibiotics. Since the signs and symptoms of bacterial pneumonia can mimic that of a viral infection and other non-infectious pulmonary pathologies, thousands are given antibiotics unnecessarily. This leads to increased health care costs, exposes patients to potentially serious side effects of antimicrobial therapy, and contributes to the development of multi-drug resistant organisms. Multi-drug resistant bacteria are a burgeoning problem throughout the world. For example, drug resistant Streptococcus pneumoniae has become an increasingly common cause of pneumonia, with greater than 50% of isolates in regions of Eastern Asia being penicillin resistant. Therefore, having a rapid and inexpensive method of detecting bacterial lower respiratory tract infections will help clinicians appropriately select those patients requiring antibiotic therapy.

Meningitis is an infection of the covering of the brain and spinal cord and is most commonly caused by either bacteria or viruses. Early treatment of bacterial meningitis improves patient outcomes but traditional methods of diagnosing meningitis are time consuming, not widely available and often inaccurate. While the treatment for most types of viral meningitis is supportive care, the treatment for bacterial meningitis involves the rapid initiation of broad spectrum antibiotics. The diagnosis of bacterial meningitis is challenging as the classic clinical triad of fever, neck stiffness, and altered mental status has a low sensitivity (about 44%). Consequently, lumbar puncture is central to the diagnosis of meningitis. The cerebro-spinal fluid (CSF) obtained from a lumbar puncture is typically sent for gram stain and culture and studied for levels of protein, glucose and white blood cells. The profile generated from these studies provides crucial information in diagnosing meningeal infection. However, these studies are time consuming, expensive and not always available in local hospital laboratories. For example, bacterial cultures can require 24-72 hours. While a gram stain provides a relatively rapid answer, it has a reported sensitivity as low as 60%.

When local hospitals do not have the facilities to handle CSF analysis, patients are often transferred to larger referral centers. Studies have demonstrated that rapid diagnosis has major clinical advantages. Even with appropriate therapy, mortality rates for bacterial meningitis can be as high as 40% with up to 37% of survivors sustaining permanent neurologic deficits, including hearing loss and cognitive deficits. Further complicating accurate diagnosis of bacterial meningitis, clinical and laboratory values associated with viral meningitis often mimic that of their bacterial counterparts. Complex and expensive molecular biology assays such as polymerase chain reaction (PCR) are often the only way to accurately differentiate between the two microbial causes.

Given the diagnostic challenges and the need to initiate therapy in a timely manner, many clinicians often start patients on broad spectrum antibiotics before obtaining conclusive evidence for bacterial meningitis. These antibiotics are often discontinued before a course is completed if suspicion for bacterial meningitis declines, contributing to the development of multi-drug resistant bacteria. In Western countries, vancomycin with a third generation cephalosporin has now become standard of care due to penicillin resistant organisms. Access to these newer antibiotics in the developing world is limited. In addition, initiating antibiotic therapy on all suspected individuals unnecessarily exposes uninfected patients to significant medication toxicities. Furthermore, antibiotics are often initiated prior to CSF sampling, preventing the isolation of the causative microbe and subsequent diagnosis of bacterial meningitis. In these circumstances, clinicians are forced to decide, without objective data, the treatment course and its duration.

According to a 1997 survey, urinary tract infection (UTI) accounted for nearly 7 million office visits and 1 million emergency department visits, resulting in 100,000 hospitalizations in the United States (Foxman, B (2003). Disease-a-month 49 (2): 53-70). UTI is an infection that can happen anywhere along the urinary tract, including bladders, kidneys, ureters, and urethra. The most common organism implicated in UTI (80-85%) is E. coli (Nicolle (2008). Urol Clin North Am 35 (1): 1-12). Bladder infections are most common in young women, with 10% of women getting an infection yearly and 60% having an infection at some point in their life. (Id.). UTIs may be confirmed via urinalysis (Id.). Urine culture showing a quantitative count of greater than or equal to 103 colony-forming units (CFU) per mL of a typical urinary tract organism is useful to guide antibiotic choice. (Id.).

Bacteria can infect joints, either by attacking a joint directly or by spreading form other parts of the body to the joint. In septic arthritis, bacteria infiltrate a joint and damage it, causing severe pain, warmth and swelling. Bacteria most commonly target the knee, though other joints, including ankle, hip, wrist, elbow and shoulder, may also be affected by septic arthritis. To discover the source of the infection, a sample of the fluid within the joint (synovial fluid) is taken through a needle inserted in the space around the joint. Synovial fluid normally appears clear and thick. Bacterial infections may alter the color, consistency, volume, and makeup of the synovial fluid. Lab analysis of the synovial fluid may be used to determine which organism is causing the infection. Such lab analyses are important in guiding the choice of treatment using antibiotics.

Infections associated with prosthetic-joints account for a substantial proportion of healthcare expenditures. (Zimmerli et al., (2004) New Engl. J. Med., 351:1645-54). The most commonly cultured microorganisms are coagulase-negative staphylococci (in 30 to 43 percent of cases) and Staphylococcus aureus (12 to 23 percent). (Id.) Because long-term therapy using an antimicrobial agents is commonly used in North America (Id.), accurate diagnosis of the infection-causing organism is important in finding the right treatment to eradicate the infection.

In view of the foregoing, there is a need for rapid and inexpensive methods for the detection of microbial infections, including pneumonia, meningitis, UTI, joint infections, and the like. The present invention is directed to meeting these and other needs.

### SUMMARY OF THE INVENTION

Accordingly, one embodiment of the present invention is a method for detecting whether a subject has an infection. This method comprises:

(a) incubating a test sample from a subject suspected of having an infection with a labeled molecule that is preferentially incorporated into a pathogenic microorganism for a period of time sufficient for the pathogenic microorganism to incorporate the labeled molecule;

(b) removing any unincorporated labeled molecule from the test sample; and

(c) detecting the labeled molecule within the pathogenic microorganism, if any, in the test sample, wherein the presence of labeled molecule within the pathogenic microorganism indicates that the subject has an infection.

Another embodiment of this invention is a method for detecting whether a subject has an infection. This method comprises:

(a) incubating a test sample from a subject suspected of having an infection with a labeled nucleoside analog that is preferentially phosphorylated by a thymidine kinase from a pathogenic microorganism for a period of time sufficient to phosphorylate the labeled nucleoside analog by the thymidine kinase of the pathogenic microorganism;

(b) removing any unphosphorylated or unsequestered labeled nucleoside analog from the test sample; and

(c) detecting labeled nucleoside analog sequestered in the pathogenic microorganism, if any, in the test sample, wherein the presence of labeled nucleoside analog sequestered in the pathogenic microorganism indicates that the subject has an infection.

An additional embodiment of this invention is a method for detecting the presence of a pathogenic microorganism in a test sample. This method comprises:

(a) contacting the test sample with a labeled nucleoside analog that is preferentially phosphorylated by a thymidine kinase of the pathogenic microorganism, if any, in the sample; and

(b) detecting whether the labeled nucleoside analog has been phosphorylated or incorporated into the pathogenic microorganism.

Yet another embodiment of this invention is a kit for detecting the presence of a pathogenic microorganism in a test sample. This kit comprises:

(a) a labeled nucleoside analog that is preferentially phosphorylated by a thymidine kinase of a pathogenic microorganism;

(b) a container for contacting the labeled nucleoside analog with a test sample suspected of containing the pathogenic microorganism; and

(c) a device for capturing the pathogenic microorganism, if any, after contacting the labeled nucleoside analog with the sample in the container.

An additional embodiment of this invention is a method for determining whether a subject is a candidate for antibiotic treatment. This method comprises:

(a) incubating a test sample from a subject suspected of having an infection with a labeled nucleoside analog that is preferentially phosphorylated by a thymidine kinase from a pathogenic microorganism;

(b) removing any unphosphorylated or unsequestered labeled nucleoside analog from the test sample; and

(c) detecting, in the test sample, labeled nucleoside analog sequestered in the pathogenic microorganism, if any, wherein the presence of labeled nucleoside analog sequestered in the pathogenic microorganism indicates that the subject has an infection and is a candidate for antibiotic treatment.

Another embodiment of this invention is a method for monitoring the efficacy of an antibiotic treatment in a subject in need thereof. This method comprises:

(a) incubating a test sample from a subject receiving antibiotic treatment with a labeled nucleoside analog that is preferentially phosphorylated by a thymidine kinase of a pathogenic microorganism;

(b) removing any unphosphorylated or unsequestered labeled nucleoside analog from the test sample; and

(c) detecting, in the test sample, labeled nucleoside analog sequestered in the pathogenic microorganism, if any, wherein the presence and intensity, if any, of incorporated labeled nucleoside analog sequestered in the pathogenic microorganism provides an indication of the efficacy of the antibiotic treatment.

Yet another embodiment of this invention is a method for detecting bacterial or herpes simplex virus (HSV) meningitis in a subject suspected of having such a disease. This method comprises:

(a) incubating a sample of cerebro-spinal fluid from the subject with ¹⁴C-labeled 2′-fluoro-5-iodo-1-β-D-arabinofuranosyluracil (FIAU) that is preferentially phosphorylated by a thymidine kinase of a pathogenic microorganism;

(b) removing any unphosphorylated or unsequestered ¹⁴C-labeled FIAU from the sample of cerebro-spinal fluid; and

(c) detecting, in the cerebro-spinal fluid, ¹⁴C-labeled FIAU sequestered in the pathogenic microorganism, if any, wherein the presence of incorporated ¹⁴C-labeled FIAU sequestered in the pathogenic microorganism indicates that the subject has bacterial or HSV meningitis.

An additional embodiment of this invention is a kit for detecting bacterial or HSV meningitis in a subject suspected of having such a disease. This kit comprises:

(a) ¹⁴C-labeled FIAU;

(b) a container for contacting the ¹⁴C-labeled FIAU with a sample of cerebro-spinal fluid from the subject; and

(c) a device for capturing a pathogenic microorganism containing ¹⁴C-labeled FIAU from the sample, if any.

Yet another embodiment of this invention is a method for detecting bacterial pneumonia in a subject suspected of having such a disease. This method comprises:

(a) incubating a sputum sample from the subject with ¹⁴C-labeled FIAU that is preferentially phosphorylated by a thymidine kinase of a pathogenic microorganism;

(b) removing any unphosphorylated or unsequestered ¹⁴C-labeled FIAU from the sputum sample; and

(c) detecting, in the sputum sample, ¹⁴C-labeled FIAU sequestered in the pathogenic microorganism, if any, wherein the presence of ¹⁴C-labeled FIAU sequestered in the pathogenic microorganism indicates that the subject has bacterial pneumonia.

An additional embodiment of this invention is a kit for detecting bacterial pneumonia in a subject suspected of having such a disease. This kit comprises:

(a) ¹⁴C-labeled FIAU;

(b) a container for contacting the ¹⁴C-labeled FIAU with a sputum sample from the subject; and

(c) a device for capturing a pathogenic microorganism containing ¹⁴C-labeled FIAU from the sample, if any.

Yet another embodiment of the present invention is a method for detecting whether a subject has a bacterial infection and, if so, for determining whether an antibiotic has an effect on a pathogenic microorganism causing the bacterial infection. This method comprises:

(a) incubating a test sample from a subject suspected of having a bacterial infection with a labeled nucleoside analog that is preferentially incorporated into a pathogenic microorganism in the presence and in the absence of an antibiotic for a period of time sufficient for the pathogenic microorganism to incorporate the labeled nucleoside analog;

(b) removing any unincorporated labeled nucleoside analog from the test sample;

(c) detecting the labeled nucleoside analog within the pathogenic microorganism, if any, in the test sample, wherein the presence of labeled nucleoside analog within the pathogenic microorganism indicates that the subject has an infection; and

(d) comparing the amount of labeled nucleoside analog in the pathogenic microorganism incubated in the presence and in the absence of the antibiotic, wherein a reduction in the amount of labeled nucleoside analog in the pathogenic microorganism in the presence of the antibiotic indicates that the antibiotic has an effect on the pathogenic microorganism.

Another embodiment of the present invention is a method for detecting whether a subject has a bacterial infection and, if so, for selecting a treatment for the infection. This method comprises:

(a) incubating a test sample from a subject suspected of having a bacterial infection with a labeled nucleoside analog that is preferentially incorporated into a pathogenic microorganism in the presence and in the absence of an antibiotic for a period of time sufficient for the pathogenic microorganism to incorporate the labeled nucleoside analog;

(b) removing any unincorporated labeled nucleoside analog from the test sample;

(c) detecting the labeled nucleoside analog within the pathogenic microorganism, if any, in the test sample, wherein the presence of labeled nucleoside analog within the pathogenic microorganism indicates that the subject has an infection; and

(d) comparing the amount of labeled nucleoside analog in the pathogenic microorganism incubated in the presence and in the absence of the antibiotic, wherein a reduction in the amount of labeled nucleoside analog in the pathogenic microorganism in the presence of the antibiotic indicates that the antibiotic is a candidate drug for treating the bacterial infection.

Another embodiment of the present invention is a method for detecting the presence of a pathogenic bacteria in a test sample and, if such a pathogenic bacteria is present in the test sample, for identifying a candidate antibiotic for treating or ameliorating the effects of the pathogenic bacteria. This method comprises:

(a) contacting the test sample with a labeled nucleoside analog that is preferentially phosphorylated or incorporated by a thymidine kinase of the pathogenic bacteria;

(b) further contacting the test sample of step (a) with a panel of antibiotics including one or more controls;

(c) detecting whether the labeled nucleoside analog has been phosphorylated or incorporated into the pathogenic bacteria; and

(d) comparing the amount of phosphorylated or incorporated labeled nucleoside analog in the pathogenic bacteria exposed to each antibiotic or control in the panel and identifying the antibiotic from the panel, which corresponds to the largest reduction in the amount of phosphorylated or incorporated labeled nucleoside analog in the pathogenic bacteria, wherein the antibiotic identified in step (d) indicates the nature of the bacteria causing the infection and provides an antibiotic selectivity profile to a medical professional.

An additional embodiment of the present invention is a kit for detecting the presence of a pathogenic bacteria in a test sample and for determining whether an antibiotic has an effect on the pathogenic bacteria. This kit comprises:

(a) a labeled nucleoside analog that is preferentially phosphorylated by a thymidine kinase of a pathogenic bacteria;

(b) a panel of antibiotics, including a control;

(c) a container for contacting the labeled nucleoside analog with a test sample suspected of containing the pathogenic bacteria and the panel of antibiotics, including a control; and

(d) a device for capturing the pathogenic bacteria, if any, after contacting the labeled nucleoside analog with the sample in the container.

### BRIEF DESCRIPTION OF THE DRAWINGS

FIG. 1 shows that a Nytran SPC filter (pore size: 0.45 μm) preferentially binds phosphorylated FIAU over FIAU. The data shown are collected under cell-free conditions. Specifically, 1.76 μM ¹⁴C-FIAU or phosphorylated ¹⁴C-FIAU (¹⁴C-FIAU-P) in 2 ml of PBS buffer was passed through a Nytran SPC filter using a syringe. One set of filters was removed from the filter cassette holders and counted using a scintillation counter. Another set of filters was washed with 30 ml of PBS via a syringe before counting with a scintillation counter.

FIG. 2 shows that signal from 10,000 cells (DH5α E. coli) is detectable above background after 15 hours of incubation in LB media. Nytran N filter (pore size: 0.2 μm) was used.

FIG. 3 shows that signal from 10,000 cells is detectable above background after 8 hours of incubation in LB media.

FIG. 4 shows that Millipore's Nitro Cellulose Membrane (NCM) Filter has the least non-specific binding to ¹⁴C-FIAU in comparison to the Nytran N filter or PALL Supor200 filter.

FIG. 5 shows that 10,000 cells incubated in LB or SOB media for 8 hours provided strong signal when tested using Millipore NCM filter.

FIG. 6 shows that 10,000 cells incubated in SOB for 5 hours is detectable over the background.

FIG. 7 shows that 10,000 cells incubated in 50% CSF and 50% SOB for 8 hours is detectable over the background.

FIG. 8 shows that 10,000 cells incubated in 50% urine and 50% SOB for 8 hours is detectable over the background.

FIG. 9 shows that 100,000 cells in urine and SOB media are instantly detectable without incubation.

FIG. 10 shows the results of an optimization study in which 10,000, 50,000 and 100,000 cells were incubated with 1.76 μM (200 mCi/mmol) ¹⁴C-FIAU for 4-6 hours.

FIG. 11 shows the dose dependent variation of signal for FIAU. Various concentrations of ¹⁴C-FIAU (200 mci/mmol) were incubated with bacteria for 4 hours.

FIG. 12 shows that 10,000 DH5α (E. coli) are detectable in a 50% cerebral spinal fluid (CSF) and 50% SOB mix in 5 hours.

FIG. 13 shows that 10,000 DH5α (E. coli) are detectable in a 50% urine and 50% SOB mix in 5 hours.

FIG. 14A shows the signal from urinary tract infection (UTI) positive donors normalized with respect to background. A signal that is 2× above background is considered to be positive. In this assay, the false negative (arrows) rate is 35.29%. FIG. 14B shows the signal from UTI negative donors normalized with respect to background. A signal that is 2× above background is considered to be positive. In this assay, the false positive (arrows) rate is 21.42%. Please note that for the actual experiments, donor samples were evaluated in a blinded fashion. For the ease of illustration, however, the UTI positive and negative samples were plotted separately.

### DETAILED DESCRIPTION OF THE INVENTION

One embodiment of this invention is a method for detecting whether a subject has an infection. This method comprises:

(a) incubating a test sample from a subject suspected of having an infection with a labeled molecule that is preferentially incorporated into a pathogenic microorganism for a period of time sufficient for the pathogenic microorganism to incorporate the labeled molecule;

(b) removing any unincorporated labeled molecule from the test sample; and

(c) detecting the labeled molecule within the pathogenic microorganism, if any, in the test sample, wherein the presence of labeled molecule within the pathogenic microorganism indicates that the subject has an infection.

As used herein, a “subject” is an animal, preferably, a mammal, such as, a human. In addition to humans, categories of animals within the scope of the present invention include, for example, agricultural animals, domestic animals, laboratory animals, etc. Some examples of agricultural animals include cows, pigs, horses, goats, chicken, etc. Some examples of domestic animals include dogs, cats, birds, etc. Some examples of laboratory animals include rats, mice, rabbits, guinea pigs, etc.

As used herein, an “infection” means invasion and multiplication of another organism, such as, e.g., a pathogenic microorganism, in the subject. The infection may be a bacterial infection or a viral infection. For example, the infection may be bacterial meningitis, viral meningitis such as herpes simplex virus (HSV) meningitis, bacterial pneumonia, urinary tract infection, an infection associated with a prosthetic, abscess, bacterial blood infection, bacterial periotonitis, bacterial pulmonary effusion, skin infection, soft tissue infection, post-surgical infection, or an infection related to implanted, non-prosthetic, hardware.

As used herein, a “test sample” means a specimen. In the present invention, a test sample may be obtained from, e.g., a food source or from the subject, as indicated by the context. If obtained from the subject as in the present embodiment, the test sample may be a body fluid, or body tissue. Non-limiting examples of body fluids include whole blood, serum, plasma, interstitial fluid, saliva, ocular lens fluid, cerebro-spinal fluid, sweat, urine, stool, milk, ascites fluid, mucous, nasal fluid, sputum, synovial fluid, peritoneal fluid, vaginal fluid, menses, amniotic fluid, semen, gastric juice, vomit, lymph, and post-operative fluid collections. Preferably, the body fluid is cerebro-spinal fluid or sputum. Non-limiting examples of body tissue include a biopsy and a scraping. The biopsy may be a bone or organ biopsy. The scraping may be a skin or mucosal scraping.

As used herein, a “labeled” molecule means a molecule that is tagged such that it is identifiable or traceable. The label may be a radioactive label, an enzymatic label, a chemiluminescent label, a fluorescent label, or other well known labels. Methods of labelling molecules are known in the art. Generally, radioactive labels are generated by replacing an atom with one of a distinctive radioactive isotope. Enzymatic labels, chemiluminescent labels, and fluorescent labels may be generated by attaching one or more enzymes, fluorescent dyes, or other compounds to the molecule. Enzymatic labels may be attached, e.g., using residue-specific chemistries, such as, through cysteine residues (Levine et al., J. Am. Chem. Soc. 1978, 100, 7670-7677; and Kaiser et al., Science 1984, 226, 505-11), lysine residues (McFarland et al., J Am Chem Soc 2005, 127, 13490-1), tyrosine residues (Tilley et al., J Am Chem Soc 2006, 128, 1080-1; Joshi et al., J Am Chem Soc 2004, 126, 15942-3), and tryptophan residues (Antos et al., J Am Chem Soc 2004, 126, 10256-7). Methods of fluorescent labeling are disclosed in e.g., U.S. Pat. Nos. 5,583,236 and 7,541,454. Methods of chemiluminescent labeling are disclosed in e.g., U.S. Pat. No. 4,946,958. Radioactive labeling of nucleoside analogs are disclosed in e.g., Mangner et al., Nucl Med. Biol. 2003 April; 30(3):215-24; Jacobs et al., J Nucl

Med. 2001, 42(3):467-75; Anderson et al., Nucl Med. Biol. 2010 37(4):439-42; Cai et al., Nucl Med. Biol. 2011 July; 38(5):659-66, and U.S. patent application Ser. No. 10/831,626.

The labeled molecule may be a labeled nucleoside analog, a labeled non-nucleoside analog, or mixtures thereof. Nucleosides consist of a base covalently attached to a sugar group, such as a ribose or deoxyribose, via a beta-glycosidic linkage. In RNA, the sugar is a ribose, and in DNA, the sugar is a deoxyribose, i.e., a sugar lacking a hydroxyl group that is present in ribose. The base may be a purine or a pyrimidine. Naturally occurring purines are adenine (A) and guanine (G), and the pyrimidines are cytosine (C) and thymine (T) (or in the context of RNA, uracil (U)). Examples of nucleosides include cytidine, uridine, adenosine, guanosine, thymidine. Nucleosides can be phosphorylated by kinases in the cell on the hydroxyl group attached to, e.g., the C-5 of the sugar, producing nucleotides, which are the molecular building-blocks of DNA and RNA. Nucleotides are usually mono, di- or triphosphates.

As used herein, a “nucleoside analog” means a molecule that resembles a naturally occurring nucleoside, but which has a chemical or physical modification on the base and/or the sugar moiety, such as a different or additional side group. Such analogs are discussed in, e.g., Scheit, Nucleotide Analogs (John Wiley & Son, 1980) and Uhlman et al., Chemical Reviews 90:543-584, 1990. Non-limiting examples of nucleoside analogs according to the present invention include 2′-fluoro-5-iodo-1-β-D-arabinofuranosyluracil (FIAU), 2′-fluoro-5-methyl-1-β-D-arabinofuranosyluracil (FMAU), 2′-fluoro-5-fluoro-1-β-D-arabinofuranosyuracil (FFAU), 2′-fluoro-5-iodo-1-β-D-arabinofuranosyl-cytosine (FIAC), 2′-fluoro-1-β-D-arabinofuranosyluracil (FAU), 1-(2′deoxy-2′-fluoro-(3-D-arabinofuranosyl)-5-ethyluracil (FEAU), 3′-deoxy-3′-fluorothymidine (FLT), 9-[4′-fluoro-3′-(hydroxymethyl)butyl]guanine (FHBG), 9-[(3′-fluoro-1′-hydroxy-2′-propoxy)methyl]guanine (FHPG), 2-amino-9-[4-hydroxy-3-(hydroxymethyl)butyl]-6,9-dihydro-3H-purin-6-one (penciclovir), 2-amino-9-((2-hydroxyethoxy)methyl)-1H-purin-6(9H)-one (acyclovir), 2-amino-9-{[(1,3-dihydroxypropan-2-yl)oxy]methyl}-6,9-dihydro-3H-purin-6-one (ganciclovir), and combinations thereof. Preferably, the labeled nucleoside analog is a radio-labeled FIAU (r-FIAU), such as a ¹²⁴I-, a ¹²⁵I-, a ¹⁸F-, or a ¹⁴C-labeled FIAU.

As used herein, a “non-nucleoside analog” means a compound that is not a nucleoside analog, but selectively targets the pathogenic microorganism rather than the subject. A non-nucleoside analog may be an antibiotic, which targets bacteria. Suitable antibiotics include an aminoglycoside, an ansamycin, a carbacephem, a carbapenem, a cephalosporin, a glycopeptide, a lincosamide, a lipopeptide, a macrolide, a monobactam, a nitrofuran, a penicillin, a penicillin combination drug, a polypeptide, a quinolone, a sulphonamide, a polycyclic naphthacene carboxamide derivative, an anti-mycobacteria drug, an unclassified conventional antibacterial drug, and combinations thereof.

As used herein, an “aminoglycoside” means an antibiotic, which or a portion of which is composed of amino-modified sugars. Non-limiting examples of aminoglycosides include Amikacin, Gentamicin, Kanamycin, Neomycin, Netilmicin, Tobramycin, Paromomycin, pharmaceutically acceptable salts thereof, and combinations thereof.

As used herein, an “ansamycin” means an antibiotic that comprises an aromatic moiety bridged by an aliphatic chain. Non-limiting examples of ansamycins include geldanamycin, herbimycin, pharmaceutically acceptable salts thereof, and combinations thereof.

As used herein, a “carbacephem” means a derivative of cephalosporin, in which the sulfur atom in the dihydrothiazine ring of the cephalosporin is substituted with a methylene group to form a tetrahydropyridine ring. Non-limiting examples of carbacephem include loracarbef, pharmaceutically acceptable salts thereof, and combinations thereof.

As used herein, a “carbapenem” means a class of β-lactam antibiotics that are derivatives of thienamycin. Non-limiting examples of carbapenems include Thienamycin, Ertapenem, Doripenem, Imipenem/Cilastatin, Meropenem, pharmaceutically acceptable salts thereof, and combinations thereof.

As used herein, a “cephalosporin” means a class of β-lactam antibiotics originally obtained from Acremonium and their derivatives. Non-limiting examples of cephalosporins include Cefadroxil, Cefazolin, Cefalothin, Cefalexin, Cefaclor, Cefamandole, Cefoxitin, Cefprozil, Cefuroxime, Cefixime, Cefmetazole, Cefdinir, Cefditoren, Cefoperazone, Cefotaxime, Cefpodoxime, Ceftazidime, Ceftibuten, Ceftizoxime, Ceftriaxone, Cefepime, Cefetamet, Cefonicid, Cefotetan, Cefsulodin, Ceftobiprole, pharmaceutically acceptable salts thereof, and combinations thereof.

As used herein, a “glycopeptide” means a class of antibiotics composed of glycosylated cyclic or polycyclic nonribosomal peptides. Non-limiting examples of glycopeptides include Teicoplanin, Vancomycin, Telavancin, pharmaceutically acceptable salts thereof, and combinations thereof.

As used herein, a “lincosamide” means derivatives of lincomycin that interfere with bacterial protein synthesis. Non-limiting examples of lincosamide include Clindamycin, Lincomycin, pharmaceutically acceptable salts thereof, and combinations thereof.

As used herein, a “lipopeptide” means an antibiotic consisting of a lipid connected to a peptide. Non-limiting examples of lipopeptide include daptomycin, pharmaceutically acceptable salts thereof, and combinations thereof.

As used herein, a “macrolide” means an antibiotics with a macrocyclic lactone ring to which one or more deoxy sugars may be attached. Non-limiting examples of macrolides include Azithromycin, Clarithromycin, Dirithromycin, Erythromycin, Roxithromycin, Troleandomycin, Telithromycin, Spectinomycin, pharmaceutically acceptable salts thereof, and combinations thereof.

As used herein, “monobactam” means a β-lactam compound wherein the β-lactam ring is alone and not fused to another ring (in contrast to most other (3-lactams, which have at least two rings). Non-limiting examples of monobactam include aztreoman, pharmaceutically acceptable salts thereof, and combinations thereof.

As used herein, a “nitrofuran” means an antibiotic containing a furan ring with a nitro group. Non-limiting examples of nitrofurans include furazolidone, nitrofurantoin, pharmaceutically acceptable salts thereof, and combinations thereof.

As used herein, a “penicillin” means a group of antibiotics derived from the Penicillium fungi. Non-limiting examples of penicillins include Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, pharmaceutically acceptable salts thereof, and combinations thereof.

As used herein, a “penicillin combination drug” means an antibiotic therapy that combines penicillin with one or more other drugs to improve the efficacy of the antibiotic in the combination. Such other drug(s) may be, for example, an irreversible inhibitor of beta-lactamase. Non-limiting examples of penicillin combination drugs include Amoxicilin/clavulanate, Ampicillin/sulbactam, Piperacillin/tazobactam, Ticarcillin/clavulanate, pharmaceutically acceptable salts thereof, and combinations thereof.

As used herein, a “polypeptide” in reference to a non-nucleoside analog of the present invention means an antibiotic that is a linked sequence of amino acids, which may be natural, synthetic, or a modification or a combination of natural and synthetic. Non-limiting examples of polypeptide non-nucleoside analogs include Bacitracin, Colistin, Polymyxin B, pharmaceutically acceptable salts thereof, and combinations thereof.

As used herein, a “quinolone” means a class of synthetic broad-spectrum antibacterial drugs derived from quinoline. Quinolones include fluoroquinolones, which have a fluorine atom attached to the central ring system, typically at the 6-position or the C-7 position. Non-limiting examples of quinolones include Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Cinoxacin, Fleroxacin, pharmaceutically acceptable salts thereof, and combinations thereof.

As used herein, a “sulfonamide” means an antibiotic that contains a sulfonamide functional group, or —S(═O)₂—NH₂, a sulfonyl group connected to an amine group. Non-limiting examples of sulfonamides include Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, Trimethoprim-Sulfamethoxazole (Co-trimoxazole) (TMP-SMX), pharmaceutically acceptable salts thereof, and combinations thereof.

As used herein, a “polycyclic naphthacene carboxamide” means a polyketide antibiotic having an octahydrotetracene-2-carboxamide skeleton and its derivatives. Polycyclic naphthacene carboxamides include glycylcyclines. Non-limiting examples of polycyclic naphthacene carboxamides include Demeclocycline, Doxycycline, Minocycline, Oxytetracycline, Tetracycline, Tigecycline, pharmaceutically acceptable salts thereof, and combinations thereof.

As used herein, an “anti-mycobacteria drug” means an antibiotic used to treat diseases caused by members of the Mycobacterium genus, including tuberculosis (TB) and leprosy. Non-limiting examples of anti-mycobacteria drugs include Clofazimine, Dapsone, Capreomycin, Cycloserine, Ethambutol, Ethionamide, Isoniazid, Pyrazinamide, Rifampin, Rifabutin, Rifapentine, Streptomycin, pharmaceutically acceptable salts thereof, and combinations thereof.

As used herein, an “unclassified conventional antibacterial drug” means an antibiotic that does not fall within any of the above classes of antibiotics and that is used to treat bacterial infection. Non-limiting examples of unclassified conventional antibacterial drugs include Arsphenamine, Chloramphenicol, Fosfomycin, Fusidic acid, Linezolid, Metronidazole, Mupirocin, Platensimycin, Quinupristin/Dalfopristin, Rifaximin, Thiamphenicol, Tinidazole, pharmaceutically acceptable salts thereof, and combinations thereof.

As used herein, a molecule that is “sequestered” or preferentially “incorporated” into a pathogenic microorganism means a molecule that is selectively accumulated in a pathogenic microorganism or bound to the surface of the pathogenic microorganism. Accumulation includes uptake of the molecule into the cell, such as, e.g., the cytoplasm and/or the nucleus, and integration of the molecule into the DNA. Such preferential incorporation may occur because of differences in proteins present in the pathogenic microorganism and the subject. For instance, thymidine kinases in pathogenic microorganisms, such as viruses and bacteria, have different substrate specificity from those in mammalian subjects. Thus, thymidine kinases of pathogenic microorganisms may catalyze the reaction phosphorylating certain nucleosides and nucleoside analogs, such as those disclosed above, more efficiently. Once phosphorylated, the diffusion of these phosphorylated nucleosides and/or nucleoside analogs out of the cells is greatly reduced, and/or the phosphorylated nucleosides and/or nucleoside analogs are incorporated into the genome of these microorganisms. In the case of certain antibiotics, preferential incorporation may occur because antibiotics bind to enzymes that are specific to bacteria or disrupt certain enzymatic processes that are specific to bacteria.

As used herein, “a period of time sufficient” for the pathogenic microorganism, such as a pathogenic bacteria, to incorporate the labeled molecule means the minimum amount of time needed for the incorporation of the labeled molecules. Such a period of time may be instantaneous, or least about 30 minutes, such as at least about 45 minutes, or at least about 1, 2, 3, 4, 5, 6, 7, 8, 9, 10, 11, 12, 13, 14, 15, 16, 17, 18, 19, 20, 22, 25, 27, 30, 35, 40, 45, 50, 55, or 60 hours. Such a period of time may also be influenced by the number of cells in the sample. Preferably, the methods of the present invention are on samples having more than 10 cells, such as more than 50 cells, including more than 100 cells, more than 500 cells, more than 1,000 cells, more than 10,000 cells, more than 50,000 cells, and more than 100,000 cells. In certain embodiments, the number of cells in the sample is between 1,000 cells and about 10,000 cells. In the present invention, any combination of the above-identified time and cell ranges is contemplated.

Unincorporated labeled molecules may be removed from the test sample by, e.g., centrifugation or filtering. Suitable filters include those disclosed in the Examples. Preferably, the filters are neutral in charge.

In one aspect of this embodiment, this method further comprises removing particles larger than the pathogenic microorganism, such as, e.g., tissue debris, white blood cells, or red blood cells, prior to removing any unincorporated labeled molecule from the test sample. The removal of such large particles may be achieved by e.g., filtering. Suitable filters include those that have a pore size larger than about 2.5 μm. For example, such pre-filtering may be accomplished using a 5 μm filter.

Another embodiment of this invention is a method for detecting whether a subject has an infection, such as a bacterial or a viral infection. This method comprises:

(a) incubating a test sample from a subject suspected of having an infection with a labeled nucleoside analog that is preferentially phosphorylated by a thymidine kinase from a pathogenic microorganism for a period of time sufficient to phosphorylate the labeled nucleoside analog by the thymidine kinase of the pathogenic microorganism;

(b) removing any unphosphorylated or unsequestered labeled nucleoside analog from the test sample; and

(c) detecting labeled nucleoside analog sequestered in the pathogenic microorganism, if any, in the test sample, wherein the presence of labeled nucleoside analog sequestered in the pathogenic microorganism indicates that the subject has an infection.

As used herein, a “thymidine kinase” means an enzyme that catalyses the reaction to convert a deoxythymidine to a deoxythymidine 5′-phosphate.

As used herein, “unsequestered” with reference to a labeled nucleoside analog means a labeled nucleoside analog that is not incorporated in cells, e.g., the pathogenic microorganism.

In this embodiment, the types of infections, suitable subjects, labels, and nucleoside analogs are as set forth above. Preferably, the labeled nucleoside analog is a radio-labeled FIAU (r-FIAU), such as a ¹²⁴I-, a ¹²⁵I-, a ¹⁸F-, or a ¹⁴C-labeled FIAU.

In one aspect of this embodiment, the test sample is a body fluid or a body tissue. In this embodiment, the types of body fluid and body tissue are as set forth above. Preferably, the body fluid is cerebro-spinal fluid or sputum. The body tissue may also be a biopsy, such as a bone or organ biopsy, or a scraping, such as a skin or a mucosal scraping.

In another aspect of this embodiment, this method further comprises removing particles larger than the pathogenic microorganism, such as, e.g., tissue debris, white blood cells, or red blood cells, prior to removing any unphosphorylated or unsequestered labeled nucleoside analog from the test sample. The removal of such large particles may be achieved by e.g., filtering. Suitable filters include those that have a pore size larger than about 2.5 μm. For example, such pre-filtering may be accomplished using a 5 μm filter.

An additional embodiment of this invention is a method for detecting the presence of a pathogenic microorganism in a test sample. This method comprises:

(a) contacting the test sample with a labeled nucleoside analog that is preferentially phosphorylated by a thymidine kinase of the pathogenic microorganism, if any, in the sample; and

(b) detecting whether the labeled nucleoside analog has been phosphorylated or incorporated into the pathogenic microorganism.

In this embodiment, the types of infections, suitable subjects, labels and nucleoside analogs are as set forth above. Preferably, the labeled nucleoside analog is a radio-labeled FIAU (r-FIAU), such as a ¹²⁴I-, a ¹²⁵I-, a ¹⁸F-, or a ¹⁴-labeled FIAU.

In one aspect of this embodiment, the test sample is obtained from a food source. Thus, the present invention may also be used to determine whether a particular food source is contaminated with a pathogenic microorganism. In this embodiment, “food source” is to be broadly interpreted to include any substance that may be ingested by a subject, such as, e.g., processed and non-processed foods and beverages. Non-limiting representative processed foods include preserved foods, canned foods, dried foods, frozen foods, juice concentrate, pastries, and the like. Non-limiting representative non-processed foods include meats, vegetables, grains, dairy, fruits and nuts. As noted above, “food source” also includes beverages including juices, soda, milk, alcoholic beverages, and water, including natural sources of drinking water, e.g., fresh water spring, rivers, lakes, aquifers and the like.

In another aspect of this embodiment, the test sample is obtained from a body fluid or a body tissue as previously defined.

Yet another embodiment of this invention is a kit for detecting the presence of a pathogenic microorganism in a test sample. This kit comprises:

(a) a labeled nucleoside analog that is preferentially phosphorylated by a thymidine kinase of a pathogenic microorganism;

(b) a container for contacting the labeled nucleoside analog with a test sample suspected of containing the pathogenic microorganism; and

(c) a device for capturing the pathogenic microorganism, if any, after contacting the labeled nucleoside analog with the sample in the container.

In the present invention, the “device” may be any suitable apparatus for retaining a pathogenic microorganism on a surface for subsequent assay, e.g., determining whether a labeled nucleoside or nucleoside analog has been incorporated and/or sequestered within the pathogenic microorganism. Preferably, the device has a low background in the assay selected to be used to determine whether a labeled nucleoside or nucleoside analog has been incorporated and/or sequestered within the pathogenic microorganism. By “low background”, it is meant that in the absence of cells, the use of the device gives rise to little or no signal in the selected assay, such as, e.g., the device does not preferentially bind labeled nucleoside analogs. Representative devices according to the present invention include filters, surfaces coated with antibodies against pathogenic microorganisms (such as an antibody-coated glass slide or an antibody-coated bead), and containers suitable for centrifugation (such as centrifuge tubes). In a preferred embodiment, the device is a neutrally charged nitrocellulose/acetate filter such as a NCM filter from Millipore Corp. (Massachusetts). In another preferred embodiment, a second device for the removal of particles larger than the pathogenic microorganisms, such as, e.g., tissue debris, white blood cells, and red blood cells, from the test sample prior to presenting such test sample to the device for capturing the pathogenic microorganism may be also be included. Non-limiting examples of this second device include filters having a pore size larger than about 2.5 μm, such as filters having a pore size of about 5 μm.

In one aspect of this embodiment, the kit further comprises a first reagent for washing the device to remove excess and unsequestered labeled nucleoside analog. As used herein, “excess” labeled nucleoside analog means those labeled nucleoside analogs that were not bound to cells, e.g., the pathogenic microorganisms.

The first reagent for washing the device to remove excess and unsequestered labeled nucleoside analog may be any liquid that will not lyse the cells, such as water; a buffer, such as, e.g., Phosphate Buffered Saline (PBS), Tris Buffered Saline (TBS), Tris-HCl, Phosphate Buffer (PB), AP Buffer; or cell culture media, such as LB (available commercially from e.g., Invitrogen, Carlsbad, Calif.), SOB (available commercially from e.g., United States Biological, Swampscott, Mass.), SOC (available commercially from e.g., Invitrogen), DeTox™ (available commercially from e.g., Expression Technologies, Inc., San Diego, Calif.), or TB media (available commercially from e.g., Expression Technologies, Inc.).

In another aspect of this embodiment, the kit further comprises a second reagent for use in the container for suspending the labeled nucleoside analog with the test sample suspected of containing a pathogenic microorganism.

The second reagent may be any liquid that will not lyse the cells. It may be the same as or different from the first reagent. Non-limiting examples of the second reagent include water, buffers, and cell culture media, as set forth above.

The kit may be used to detect from about 10 to more than 100,000 pathogenic microorganisms from instantaneously to about after 4 hours or more, including after about 8 hours or more, of incubation in cell culture media. For example, the kit may be used to detect 10,000 or more pathogenic microorganisms, such as, e.g., E. coli, after about 8 hours of incubation in cell culture media, such as, e.g., LB, SOB, SOC, DeTox™, or TB media. Millipore Nitro Cellulose Membrane filter may be used as the device for capturing the pathogenic microorganism, and ¹⁴C-FIAU may be used as the labeled nucleoside analog. The kit may also be able to detect phosphorylation products of FIAU by any member of the thymidine kinase family of enzymes.

An additional embodiment of this invention is a method for determining whether a subject is a candidate for antibiotic treatment. This method comprises:

(a) incubating a test sample from a subject suspected of having an infection with a labeled nucleoside analog that is preferentially phosphorylated by a thymidine kinase from a pathogenic microorganism;

(b) removing any unphosphorylated or unsequestered labeled nucleoside analog from the test sample; and

(c) detecting, in the test sample, labeled nucleoside analog sequestered in the pathogenic microorganism, if any, wherein the presence of labeled nucleoside analog sequestered in the pathogenic microorganism indicates that the subject has an infection and is a candidate for antibiotic treatment.

Another embodiment of this invention is a method for monitoring the efficacy of an antibiotic treatment in a subject in need thereof. This method comprises:

(a) incubating a test sample from a subject receiving antibiotic treatment with a labeled nucleoside analog that is preferentially phosphorylated by a thymidine kinase of a pathogenic microorganism;

(b) removing any unphosphorylated or unsequestered labeled nucleoside analog from the test sample; and

(c) detecting, in the test sample, labeled nucleoside analog sequestered in the pathogenic microorganism, if any, wherein the presence and intensity, if any, of incorporated labeled nucleoside analog sequestered in the pathogenic microorganism provides an indication of the efficacy of the antibiotic treatment.

In one aspect of this embodiment, this method further comprises removing particles larger than the pathogenic microorganism, such as, e.g., tissue debris, white blood cells, or red blood cells, prior to removing any unphosphorylated or unsequestered labeled nucleoside analog from the test sample. The removal of such large particles may be achieved by e.g., filtering. Suitable filters include those that have a pore size larger than about 2.5 μm. For example, such pre-filtering may be accomplished using a 5 μm filter.

Yet another embodiment of this invention is a method for detecting bacterial or HSV meningitis in a subject suspected of having such a disease. This method comprises:

(a) incubating a sample of cerebro-spinal fluid from the subject with ¹⁴C-labeled FIAU that is preferentially phosphorylated by a thymidine kinase of a pathogenic microorganism;

(b) removing any unphosphorylated or unsequestered ¹⁴C-labeled FIAU from the sample of cerebro-spinal fluid; and

(c) detecting, in the cerebro-spinal fluid, ¹⁴C-labeled FIAU sequestered in the pathogenic microorganism, if any, wherein the presence of incorporated ¹⁴C-labeled FIAU sequestered in the pathogenic microorganism indicates that the subject has bacterial or HSV meningitis.

In one aspect of this embodiment, this method further comprises removing particles larger than the pathogenic microorganism prior to removing any unphosphorylated or unsequestered ¹⁴C-labeled FIAU from the sample of cerebro-spinal fluid. The removal of such large particles may be achieved by e.g., filtering. Suitable filters include those that have a pore size larger than about 2.5 μm. For example, such pre-filtering may be accomplished using a 5 μm filter.

An additional embodiment of this invention is a kit for detecting bacterial or HSV meningitis in a subject suspected of having such a disease. This kit comprises:

(a) ¹⁴C-labeled FIAU;

(b) a container for contacting the ¹⁴C-labeled FIAU with a sample of cerebro-spinal fluid from the subject; and

(c) a device for capturing a pathogenic microorganism containing ¹⁴C-labeled FIAU from the sample, if any.

Suitable containers and devices are as disclosed herein. As set forth above, additional reagents and devices may also be included in the kits of the present invention.

Yet another embodiment of this invention is a method for detecting bacterial pneumonia in a subject suspected of having such a disease. This method comprises:

(a) incubating a sputum sample from the subject with ¹⁴C-labeled FIAU that is preferentially phosphorylated by a thymidine kinase of a pathogenic microorganism;

(b) removing any unphosphorylated or unsequestered ¹⁴C-labeled FIAU from the sputum sample; and

(c) detecting, in the sputum sample, ¹⁴C-labeled FIAU sequestered in the pathogenic microorganism, if any, wherein the presence of ¹⁴C-labeled FIAU sequestered in the pathogenic microorganism indicates that the subject has bacterial pneumonia.

In one aspect of this embodiment, this method further comprises removing particles larger than the pathogenic microorganism prior to removing any unphosphorylated or unsequestered ¹⁴C-labeled FIAU from the sputum sample. The removal of such large particles may be achieved by e.g., filtering. Suitable filters include those that have a pore size larger than about 2.5 μm. For example, such pre-filtering may be accomplished using a 5 μm filter.

An additional embodiment of this invention is a kit for detecting bacterial pneumonia in a subject suspected of having such a disease. This kit comprises:

(a) ¹⁴C-labeled FIAU;

(b) a container for contacting the ¹⁴C-labeled FIAU with a sputum sample from the subject; and

(c) a device for capturing a pathogenic microorganism containing ¹⁴C-labeled FIAU from the sample, if any.

Suitable containers and devices are as disclosed herein. As set forth above, additional reagents and devices may also be included in the kits of the present invention.

Yet another embodiment of the present invention is a method for detecting whether a subject has a bacterial infection and, if so, for determining whether an antibiotic has an effect on a pathogenic microorganism causing the bacterial infection. This method comprises:

(a) incubating a test sample from a subject suspected of having a bacterial infection with a labeled nucleoside analog that is preferentially incorporated into a pathogenic microorganism in the presence and in the absence of an antibiotic for a period of time sufficient for the pathogenic microorganism to incorporate the labeled nucleoside analog;

(b) removing any unincorporated labeled nucleoside analog from the test sample;

(c) detecting the labeled nucleoside analog within the pathogenic microorganism, if any, in the test sample, wherein the presence of labeled nucleoside analog within the pathogenic microorganism indicates that the subject has an infection; and

(d) comparing the amount of labeled nucleoside analog in the pathogenic microorganism incubated in the presence and in the absence of the antibiotic, wherein a reduction in the amount of labeled nucleoside analog in the pathogenic microorganism in the presence of the antibiotic indicates that the antibiotic has an effect on the pathogenic microorganism.

Another embodiment of the present invention is a method for detecting whether a subject has a bacterial infection and, if so, for selecting a treatment for the infection. This method comprises:

(a) incubating a test sample from a subject suspected of having a bacterial infection with a labeled nucleoside analog that is preferentially incorporated into a pathogenic microorganism in the presence and in the absence of an antibiotic for a period of time sufficient for the pathogenic microorganism to incorporate the labeled nucleoside analog;

(b) removing any unincorporated labeled nucleoside analog from the test sample;

(c) detecting the labeled nucleoside analog within the pathogenic microorganism, if any, in the test sample, wherein the presence of labeled nucleoside analog within the pathogenic microorganism indicates that the subject has an infection; and

(d) comparing the amount of labeled nucleoside analog in the pathogenic microorganism incubated in the presence and in the absence of the antibiotic, wherein a reduction in the amount of labeled nucleoside analog in the pathogenic microorganism in the presence of the antibiotic indicates that the antibiotic is a candidate drug for treating the bacterial infection.

In the above two embodiments of this invention, the methods further comprise identifying the antibiotic corresponding to the highest reduction in the amount of labeled nucleoside analog in the pathogenic microorganism.

Suitable subjects, labels, and nucleoside analogs are as set forth above. Preferably, the labeled nucleoside analog is a radio-labeled FIAU (r-FIAU), such as a ¹²⁴I-, a ¹²⁵I-, a ¹⁸F-, or a ¹⁴C-labeled FIAU. Methods for removing unincorporated labeled nucleoside analog are as disclosed herein. Suitable antibiotics are as disclosed above.

In the above two embodiments, the antibiotic in the incubation step comprises a panel of antibiotics. As used herein, a “panel” of antibiotics means a variety of antibiotics, preferably assembled in one set or one kit. The antibiotics may be in a convenient form for dispensing, such as disks, cartridges, tablets, caplets, or liquid suspension forms. Examples of a panel of antibiotics include those included in HardyDisks™ AST (Hardy Diagnostics, Santa Maria, Calif.). The panel of antibiotics optionally may include one or more controls for distinguishing between a real signal and, e.g., background and/or other non-specific effects.

In another aspect of the above two embodiments, the methods may further comprise removing particles larger than the pathogenic microorganism prior to removing any unphosphorylated or unsequestered ¹⁴C-labeled FIAU from the sputum sample. The removal of such large particles may be achieved by e.g., filtering. Suitable filters include those that have a pore size larger than about 2.5 μm. For example, such pre-filtering may be accomplished using a 5 μm filter.

Another embodiment of the present invention is a method for detecting the presence of a pathogenic bacteria in a test sample and, if such a pathogenic bacteria is present in the test sample, for identifying a candidate antibiotic for treating or ameliorating the effects of the pathogenic bacteria. This method comprises:

(a) contacting the test sample with a labeled nucleoside analog that is preferentially phosphorylated or incorporated by a thymidine kinase of the pathogenic bacteria;

(b) further contacting the test sample of step (a) with a panel of antibiotics including one or more controls;

(c) detecting whether the labeled nucleoside analog has been phosphorylated or incorporated into the pathogenic bacteria; and

(d) comparing the amount of phosphorylated or incorporated labeled nucleoside analog in the pathogenic bacteria exposed to each antibiotic or control in the panel and identifying the antibiotic from the panel, which corresponds to the largest reduction in the amount of phosphorylated or incorporated labeled nucleoside analog in the pathogenic bacteria, wherein the antibiotic identified in step (d) indicates the nature of the bacteria causing the infection and provides an antibiotic selectivity profile to a medical professional.

Suitable antibiotics are as disclosed herein. As used herein, “control” includes positive and/or negative controls. Preferably, the control is a negative control, in which no antibiotic is applied. In the present invention, one or more controls may be used.

As used herein, an “antibiotic selectivity profile” means a compilation of information regarding the various degrees of sensitivity of the pathogenic bacteria to different antibiotics in the panel.

In one aspect of this embodiment, this method further comprises removing particles larger than the pathogenic microorganism prior to removing any unphosphorylated or unsequestered ¹⁴C-labeled FIAU from the sputum sample. The removal of such large particles may be achieved by e.g., filtering. Suitable filters include those that have a pore size larger than about 2.5 μm. For example, such pre-filtering may be accomplished using a 5 μm filter.

An additional embodiment of the present invention is a kit for detecting the presence of a pathogenic bacteria in a test sample and for determining whether an antibiotic has an effect on the pathogenic bacteria. This kit comprises:

(a) a labeled nucleoside analog that is preferentially phosphorylated by a thymidine kinase of a pathogenic bacteria;

(b) a panel of antibiotics, including a control;

(c) a container for contacting the labeled nucleoside analog with a test sample suspected of containing the pathogenic bacteria and the panel of antibiotics, including a control; and

(d) a device for capturing the pathogenic bacteria, if any, after contacting the labeled nucleoside analog with the sample in the container.

Suitable labelled nucleoside analog, antibiotics, containers, and devices for capturing the pathogenic bacteria are as disclosed herein. As set forth above, additional reagents and devices may also be included in the kits of the present invention.

The following examples are provided to further illustrate the methods of the present invention. These examples are illustrative only and are not intended to limit the scope of the invention in any way.

### EXAMPLES

### Example 1

A nucleoside analog, Fialuridine (FIAU), is preferentially phosphorylated by and trapped within bacterial cells. The phosphorylation is catalyzed by an enzyme, thymidine kinase, that is evolutionarily conserved across bacterial species, including the difficult to detect causes of atypical pneumonia such as M. pneumonia, L. pneumophila and Coxiella burnetti. FIAU can be readily labeled with radioactive isotopes such as carbon-14 or iodine-125. Results obtained by the inventors have demonstrated that radiolabeled FIAU (r-FIAU) is taken up and retained by bacteria and may be used as an in vivo imaging agent to detect bacterial infections in humans as early as 1-2 hours after administration. Importantly, this agent is not incorporated by eukaryotic cells, including white blood cells and epithelial cells, at doses required for diagnostics.

Incorporation Of r-FIAU By Bacteria

E. coli was diluted serially in 96-well plates with Luria-Bertani (LB). Following incubation at 36° C. for 60 minutes, the cells were spun at 2500 rotations per minute for 5 minutes. The pellets were washed with 100 μl LB and spun again 2500 rotations per minute for 5 minutes. This washing process was repeated two more times. Afterwards, 25 μl of LB was added into each well, and the contents of the wells were resuspended. Then 25 μl of the bacteria-LB suspension were taken from each well and put into scinitillation fluid in 96-well format. The amount of radiation in each well was quantified. The results from one such experiment are shown in Table 1 below.

TABLE 1

| Bacterial Count | Activity |
|---|---|
| 40986000 | 474 |
| 4098600 | 424 |
| 409860 | 318 |
| 40986 | 158 |
| 4098.6 | 76 |
| 409.86 | 90 |
| LB | 25 |

### Example 2

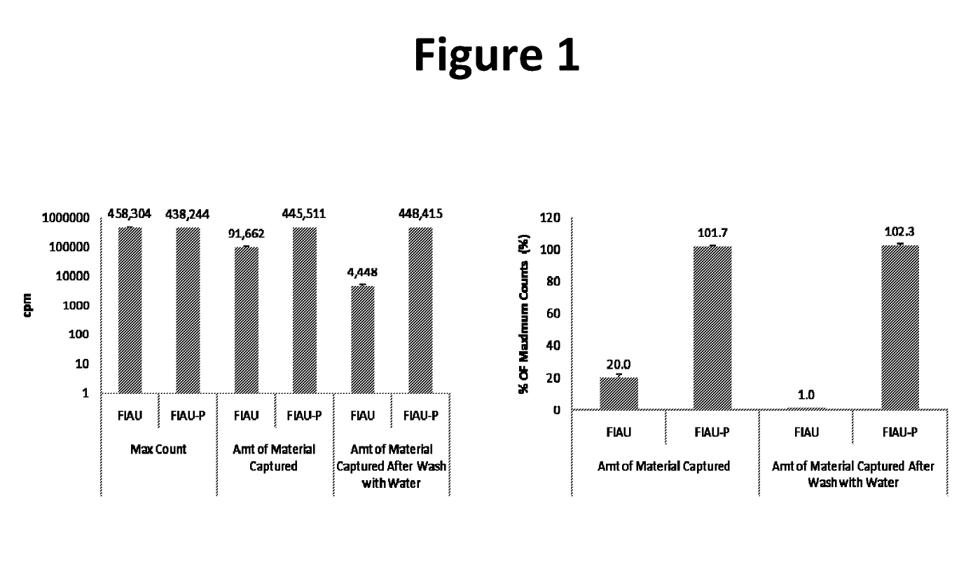

Various filters were tested for their binding to FIAU. 1.76 μM ¹⁴C-FIAU or phosphorylated ¹⁴C-FIAU (¹⁴C-FIAU-P) in 2 ml of PBS buffer was passed through a Nytran SPC filter (pore size: 0.45 μM) (Whatman Inc., Piscataway, N.J.) using a syringe. One set of filters was removed from the filter cassette holders and counted using a scintillation counter. Another set of filters was washed with 30 ml of PBS via a syringe before counting with a scintillation counter. The result of the experiment is shown in FIG. 1, which shows that the Nytran SPC preferentially binds FIAU-P over FIAU.

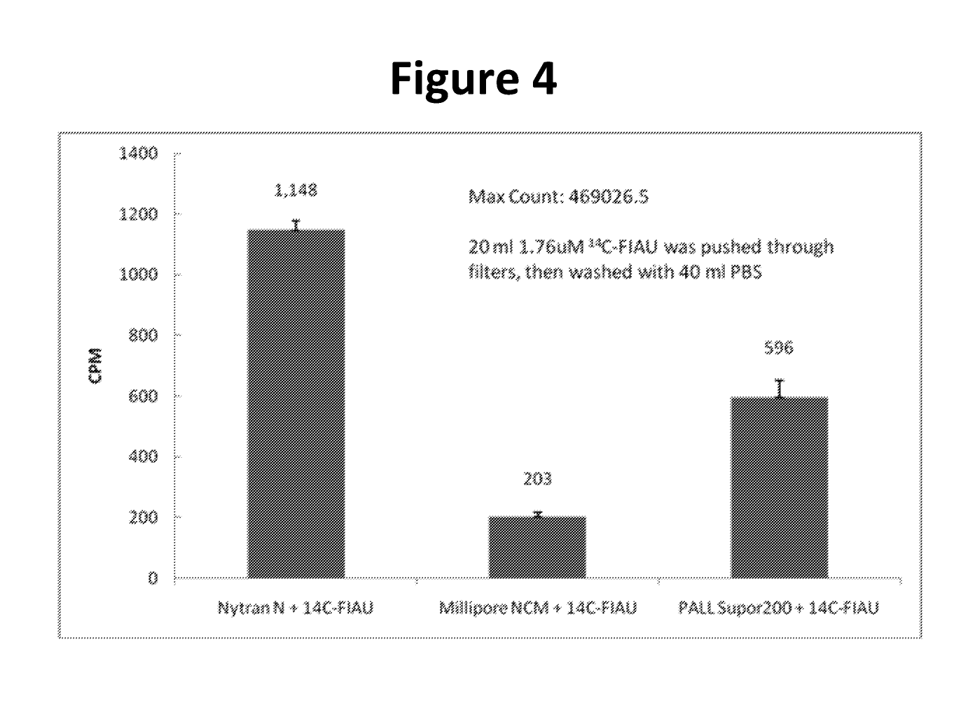

Similar experiments were conducted with Nytran N filters (pore size: 0.2 μM) (Whatman Inc.) and Millipore Nitro Cellulose Membrane (NCM) filters (pore size: 0.22 μM) (Millipore, Billerica, Mass.) and Pall Supor200 filter (pore size: 0.2 μM), (Pall Corp., Port Washington, N.Y.). Two microliters of 1.76 μM ¹⁴C-FIAU or ¹⁴C-FIAU-P in 2 ml of PBS buffer was passed through the filters using a syringe. One set of filters was removed from the filter cassette holders and counted using a scintillation counter. It was found that the Nytran N filter has low background binding of FIAU, and that the Millipore Nitro Cellulose Membrane filter has even lower non-specific FIAU binding than Nytran N filter (FIG. 4).

The Nytran SPC filter has a high positive charge, whereas the Nytran N and the NCM filters are substantially neutral in charge. Thus, filters that are substantially neutral in charge are preferred in the present invention.

### Example 3

10,000 cells were put into 2 ml of media (LB, SOB, SOC, DeTox™, or TB), and incubated for 8 hours at 37° C. and shaken at 250 rpm. The samples were then passed through the respective filters (Millipore and others) via a syringe, then the samples were washed with 40 ml of PBS via a syringe. The filter samples were counted using a scintillation counter.

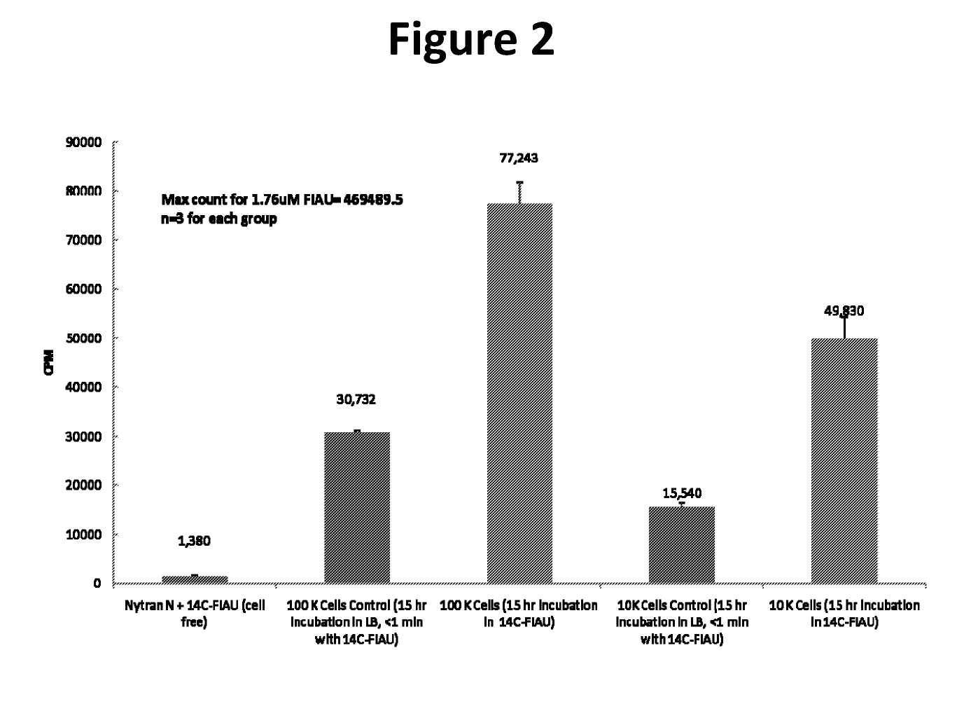

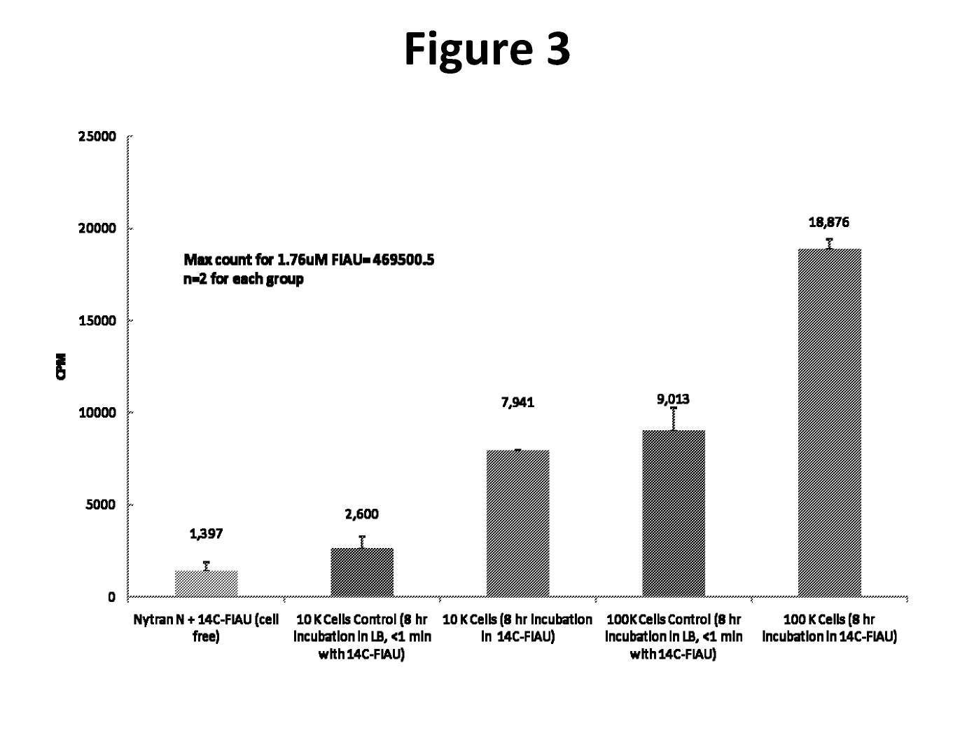

Nytran N filters were used in live cell experiments. The pore size of these filters is small enough to retain the E. coli. After eight hours of incubation of the DH5α strain of E. coli in LB media, a signal was detectable above background (FIG. 3). After 15 hours of incubation, the signal from 10,000 cells was well above background (See FIG. 2).

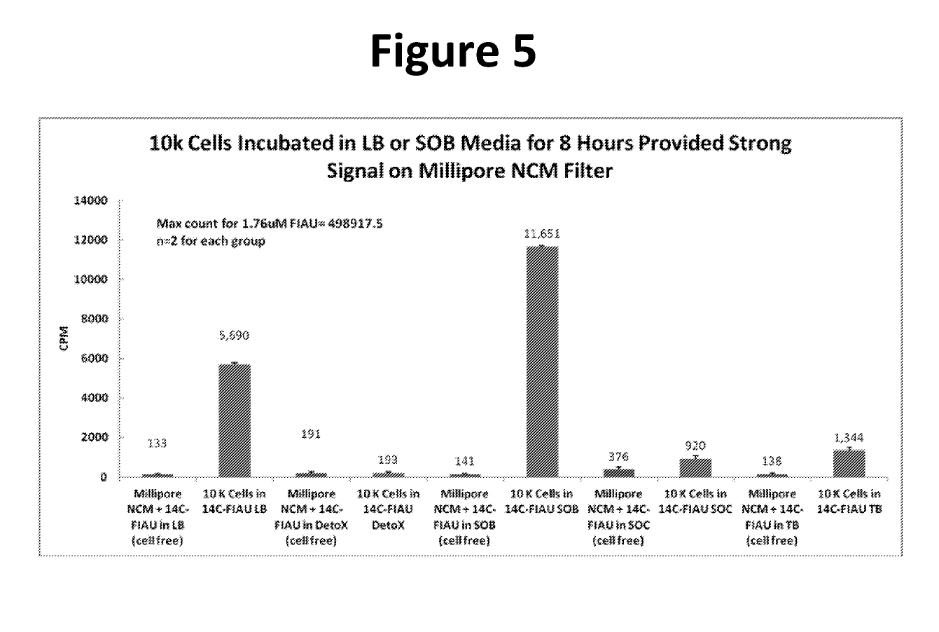

Millipore NCM filters were also used in the live cell experiments. The resulting data shows that a detectable signal above background is obtained with the Millipore NCM filter (which has very low background non-specific FIAU binding) and 10,000 cells incubated for 8 hours (FIG. 5). Moreover, LB and SOB media strongly potentiated the growth of the DH5α E. coli. The cell based signals are 42.78-fold and 82.63-fold, respectively, over background signal for these two media.

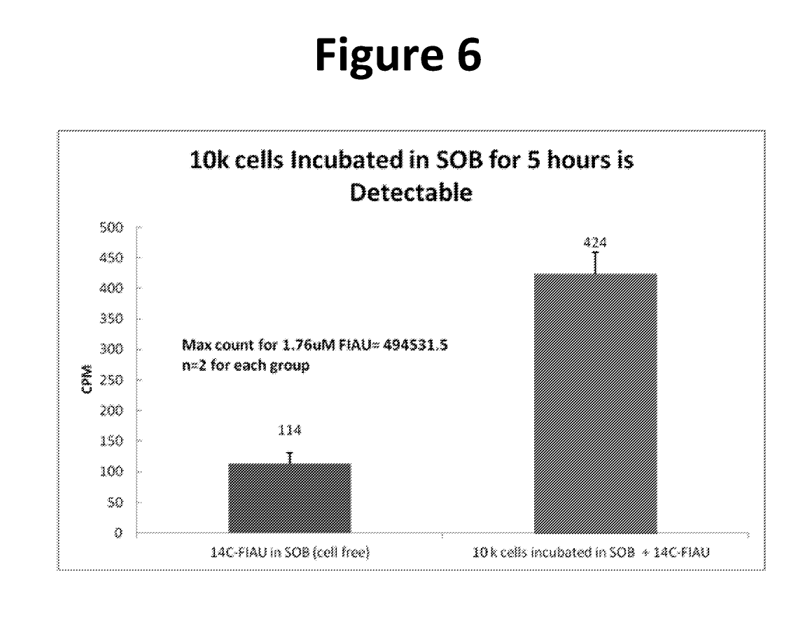

Similar experiments were conducted using 10,000 cells incubated in SOB media for 5 hours. The resulting signals were above background (FIG. 6).

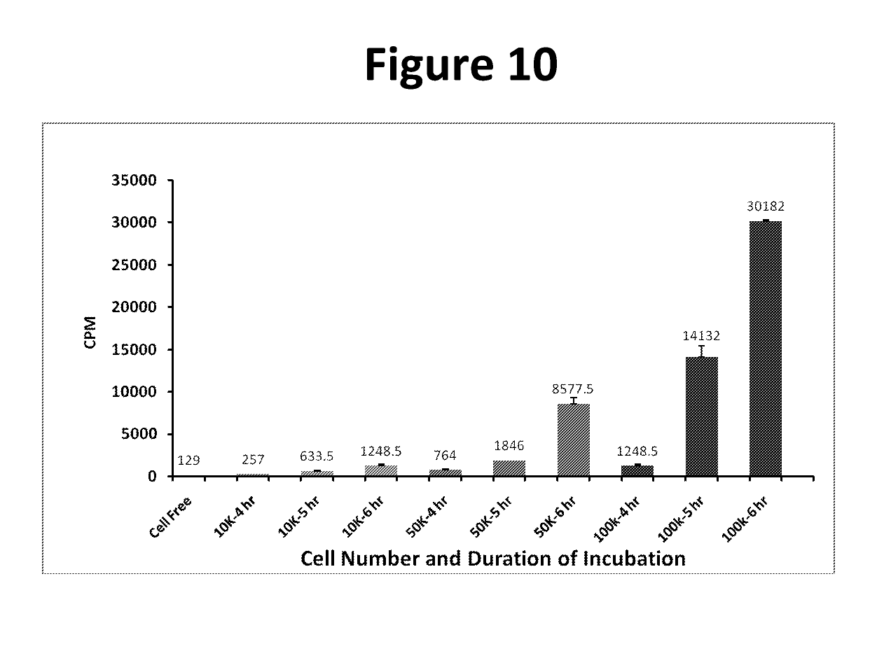

The experiments were repeated using ¹⁴C-FIAU that had a specific activity of 200 mCi/mmol. This material is 4 times more potent than the previous batch at 50 mCi/mmol. 10,000, 50,000 or 100,000 DH5α cells were added into 1 ml SOB media, and 1 μl of 1.76 mM ¹⁴C FIAU was added to the mixture to constitute a 1.76 μM solution. The mixture was incubated for 4 to 6 hours at 37° C. in an agitation incubator. After the incubation period, the mixture was passed through a 0.22 μm Millipore nitrocellulose filter. The filter was then washed with 40 ml of 1×PBS using a syringe. The CPM was determined using a scintillation counter. The results are shown in FIG. 10. 10,000 cells incubated for about 4 hours gave signal above the background.

### Example 4

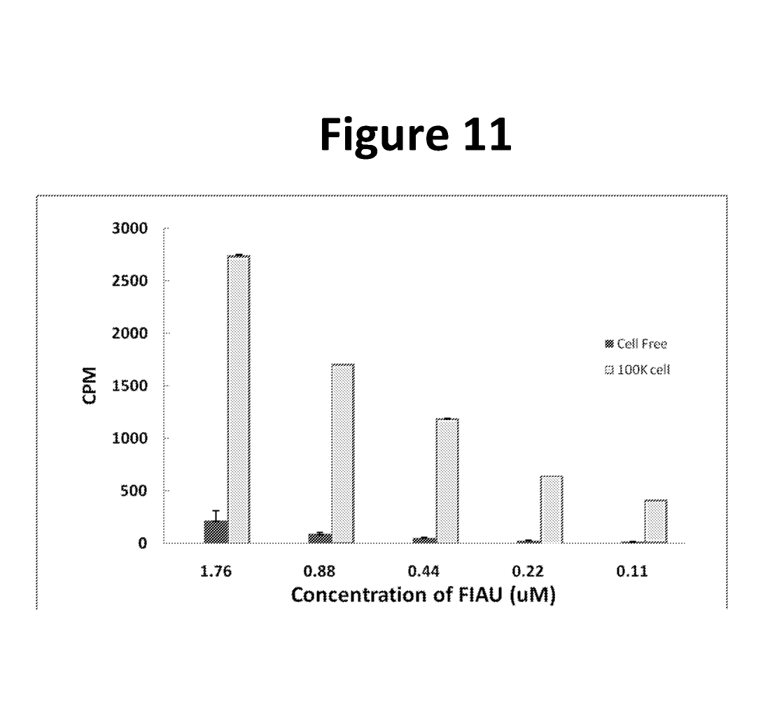

100,000 DH5α cells were added to 1 ml SOB media and various concentrations of 200 mCi/mmol ¹⁴C FIAU varying from 0.11 μM to 1.76 μM. The mixture was incubated for 4 hours at 37° C. in an agitation incubator. After the incubation period, the mixture was passed through a 0.22 μm Millipore nitrocellulose filter. The filter was then washed with 40 ml of 1×PBS using a syringe. The CPM was determined using a scintillation counter. The results are shown in FIG. 11.

### Example 5

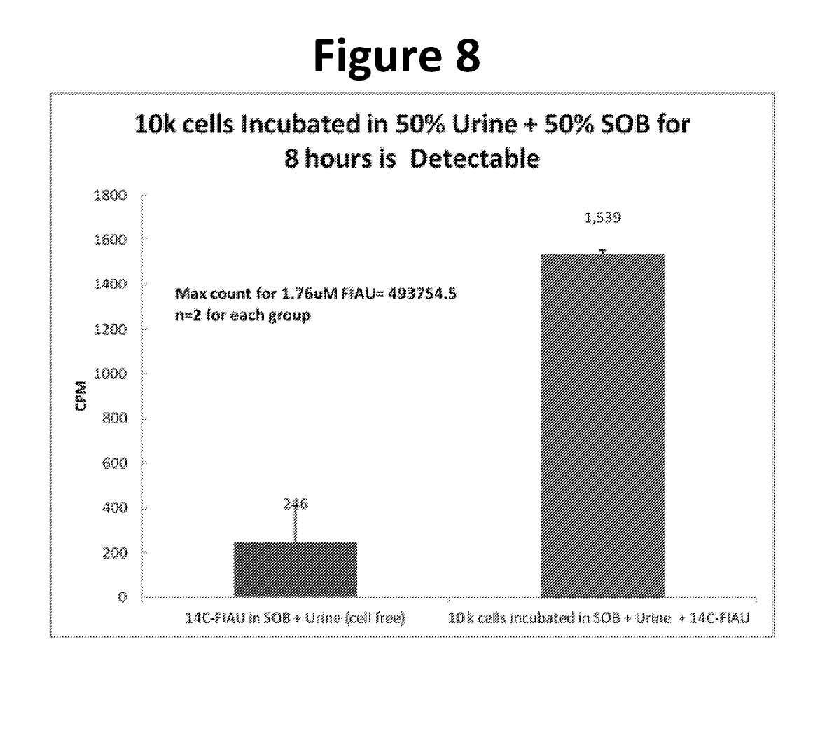

10,000 cells were incubated in a mixture of 50% urine and 50% SOB media for 8 hours. The resulting signals were well above background (FIG. 8).

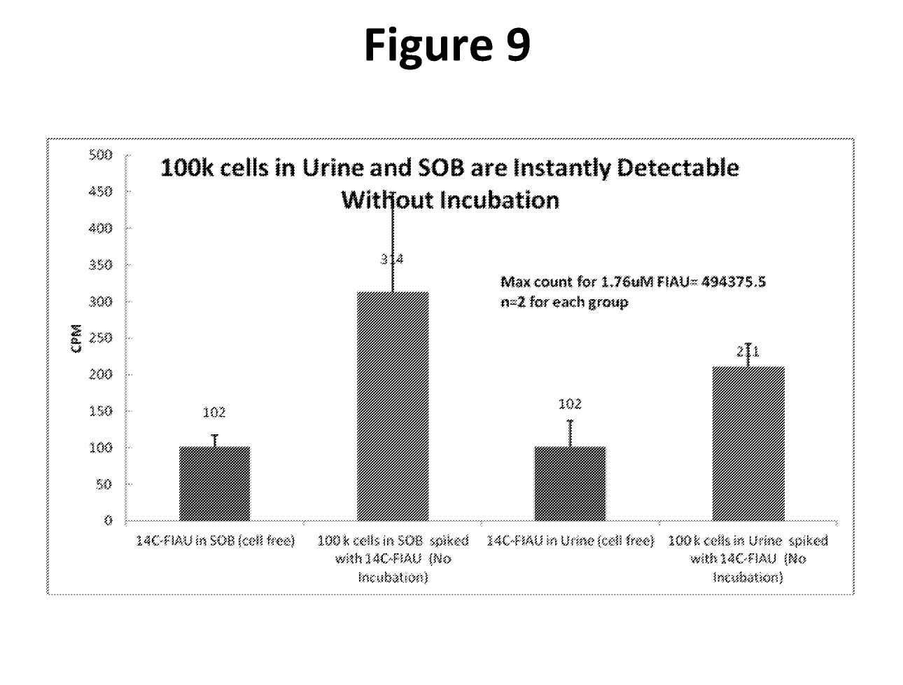

Next, 100,000 cells was spiked into 2 ml of urine sample, and 2 μl of 1.76 μM ¹⁴C-FIAU was added. The sample was immediately pushed through a 0.2 μm Millipore NCM filter via a syringe. The filter was then washed with 40 ml PBS via a syringe. The filter was removed, and the radioactivity was quantified using a scintillation counter. The results are shown in FIG. 9.

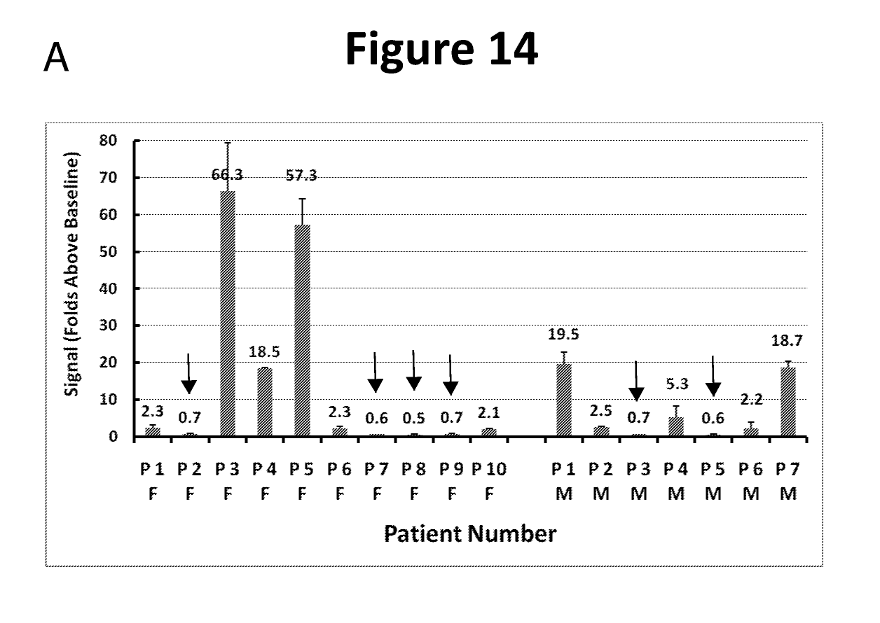

In another set of experiments, 10,000 DH5α cells were added into 1 ml human urine. 1 ml of SOB media and 2 μl of 1.76 mM ¹⁴C FIAU was added to the mixture to constitute a 1.76 μM solution. The mixture was incubated for 5 hours at 37° C. in an agitation incubator. After the incubation period, the mixture was passed through a 0.22 μm Millipore nitrocellulose filter. The filter was then washed with 40 ml of 1×PBS using a syringe. The CPM was determined using a scintillation counter. The results are shown in FIG. 13. Thus, 10,000 DH5α (E. coli) cells were detectable in a 50% urine and 50% SOB mix in 5 hours.

### Example 6

The selective uptake of FIAU by bacteria will be used to diagnose pneumonia through analysis of sputum samples. Sputum from patients suspected of having pneumonia will be incubated with trace quantities of r-FIAU. Given the preferential uptake of FIAU by bacterial cells, the bacterial load in the sputum sample is expected to correlate with radioactivity. A distinct advantage of radioactive-based diagnostic agents is the amplification of signal that is inherent to nuclear medicine technologies. Each bacterium can incorporate multiple molecules of r-FIAU, as opposed to standard culture techniques, where each bacterium is a solitary unit. In addition, while gram stains and culture data take hours-days to yield results, the assays of the present invention are expected to detect bacteria within 60 minutes. While FIAU may be labeled with a number of different radionuclides, carbon-14 (¹⁴C) is an attractive option for this indication given its 5730 year half-life and extremely benign radiation profile. As a result, ¹⁴C-FIAU has a long shelf life and can be readily stocked in any setting with no shielding. Moreover, ¹⁴C-FIAU is commercially available from, e.g., Moravek Biochemicals and Radiochemicals, Brea, Calif., and, if needed, can be synthesized in standard radiochemistry laboratories worldwide.

Normal sputum samples from 25 healthy volunteers in the United States will be tested. Each sputum sample will be incubated in the Johns Hopkins Hospital Microbiology Lab with 0.1 μCi of ¹⁴C-FIAU (Moravek Biochemicals and Radiochemicals, Brea, Calif.) or other appropriately labeled FIAU for 30-45 minutes, after which the sample will be passed through a 0.2 μm filter using a conventional syringe filtration technique. For example, the filter, which will capture bacteria and cells, is put in, e.g., a Millipore filter cassette. A luer-lok syringe with the test samples is attached to one side. Then the sample is injected into the cassette and through the filter. 40 ml of PBS will be injected the same way to rinse off the filter. The filter will be removed and counted in a scintillation counter or other similar device, such as, e.g., a high sensitivity handheld Geiger counter (Geiger, Lewiston, Me.). This will help determine the range of radioactivity seen in “normal” sputum samples. This will be especially critical when dealing with sputum, as healthy individuals have normal bacterial flora lining the respiratory tract that can contaminate samples. Gram stains will be used as quality control to ensure that the sputum samples are “adequate”. This is standard clinical practice when evaluating sputum samples and entails studying the quantity of contaminating normal flora and cells. If above a threshold, these samples will be deemed “inadequate” and will not be utilized due to an overwhelming population of normal bacteria.

Once the normal range has been established, this experiment will be further carried out in India where bacterial pneumonia is a major cause of mortality. The study will be conducted at the Manipal Hospital in Bangalore, Karnataka. ¹⁴C-FIAU will be purchased from a commercial vendor such as, e.g., Moravek Biochemicals and Radiochemicals, Brea, Calif. Governmental and institutional approval will be obtained prior to initiation of this clinical trial. Clinicians most likely to triage and treat patients with pneumonia will be educated on the trial. Sputum samples and microbiologic data from patients suspected of having pneumonia will be collected in a prospective manner. A fraction (about 5-10%) of the sample, or about 0.5-5 ml of the sample, will be incubated with ¹⁴C-FIAU and processed in the manner described above. It is expected that patients with true bacterial pneumonia will have microbes in the sputum that will incorporate r-FIAU and increase the radioactivity within the sample. If the patient does not have bacterial pneumonia, then the counts within the sputum will be within a ‘normal’ range. The goal is to define a threshold radioactivity level above which the sputum is defined as ‘positive’. If the sputum has ‘positive’ levels of ¹⁴C, then the patient may have bacterial pneumonia and may require antimicrobial therapy. If r-FIAU can stratify patients appropriately, it will allow clinicians to instantly determine whether antibiotics are indicated. This stratification can potentially save substantial cost and decrease inappropriate antibiotic use that contributes to microbial resistance. Furthermore, prompt and appropriate use of antibiotics will lead to timely recovery and reduce the morbidity and mortality associated with bacterial pneumonia.

After the trial in India, the next steps would be to perform larger scale prospective trials to determine the positive and negative predictive value of ¹⁴C-FIAU levels in diagnosing bacterial pneumonia. Response to therapy may also be monitored by testing serial sputum samples. This is important in respiratory tract infections as radiographic resolution can lag behind clinical improvement by up to 8 weeks, making the decision of when to stop antibiotics difficult.

### Example 7

r-FIAU may also be used to detect bacteria within the cerebro-spinal fluid (CSF) for diagnosing meningitis. r-FIAU has a potentially added benefit in the diagnosis of meningitis. It is phosphorylated and trapped in cells infected with the herpes simplex virus (HSV). Of the viral causes of meningitis, HSV is the one most commonly treated with anti-viral therapies. For other causes of viral meningitis, supportive therapy is generally sufficient. Therefore, r-FIAU has the potential to be preferentially incorporated by those pathogens that require anti-microbial therapy.

The selective uptake of FIAU by bacteria and HSV may be used to diagnose meningitis through analysis of CSF samples. CSF from patients suspected of having meningitis will be incubated with trace quantities of r-FIAU. Given the preferential uptake of FIAU by bacterial cells and HSV, the bacterial and HSV load in the CSF sample should correlate with radioactivity.

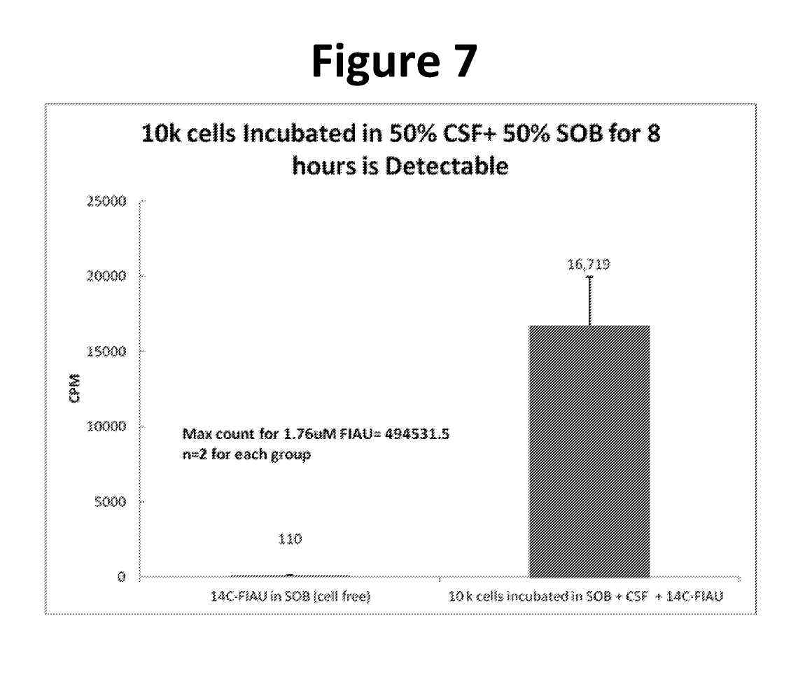

This technology was tested initially on normal CSF samples purchased from commercial vendors such as Precisionmed Inc. (San Diego, Calif.) or Biospecialty Corp. (Colmar, Pa.). Briefly, 10,000 cells were incubated in a mixture of 50% CSF and 50% SOB media for 8 hours. The resulting signals were well above background (FIG. 7).

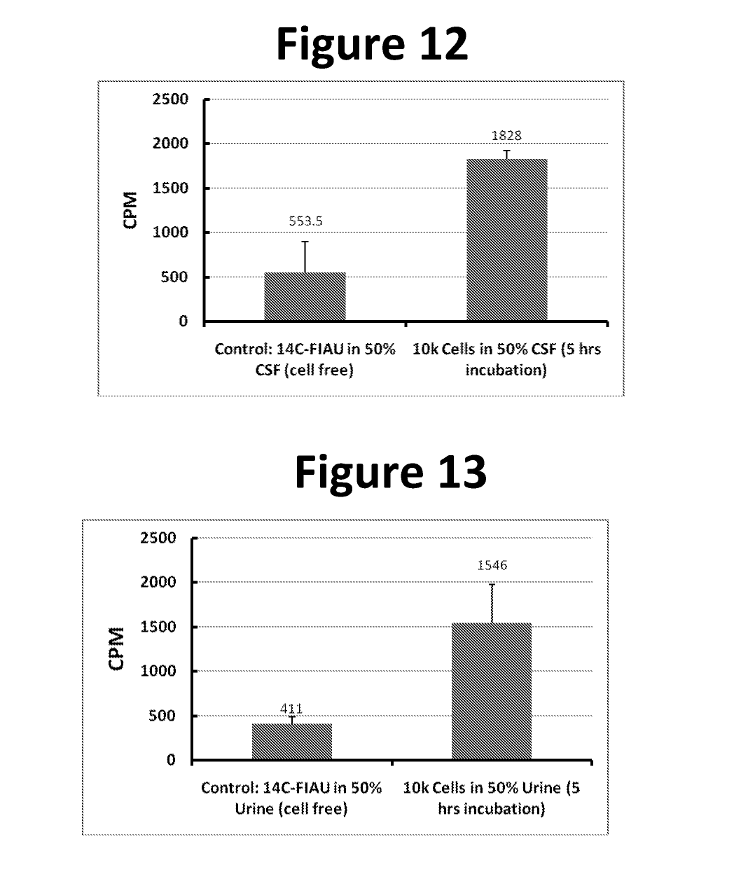

An experiment was also performed to determine whether a shorter incubation period is possible. 10,000 DH5α cells were added into 1 ml rat CSF. 1 ml of SOB media and 2 μl of 1.76 mM ¹⁴C FIAU was added to the mixture to constitute a 1.76 μM solution. The mixture was incubated for 5 hours at 37° C. in an agitation incubator. After the incubation period, the mixture was passed through a 0.22 μm Millipore nitrocellulose filter. The filter was then washed with 40 ml of 1×PBS using a syringe. The CPM was determined using a scintillation counter. The results are shown in FIG. 12. Thus, 10,000 cells after 5 hours or less of incubation in a mixture containing 50% CSF and 50% SOB were detectable.

Next, CSF samples will be spiked with known amounts of various pathogens responsible for bacterial meningitis. The sample will be incubated with 0.1 μCi of ¹⁴C-FIAU for 30-45 minutes, after which the sample will be passed through a 0.2 μm filter using the syringe filtration technique as disclosed above. The filter is expected to capture all bacteria and cells, including those infected with HSV. The excess and unbound r-FIAU will be washed away, and residual radioactivity in the filter will be quantified using a high sensitivity handheld Geiger counter or other suitable device. This will help establish a dose response curve for varying quantities of pathogens within CSF samples as well as the background rates in uninfected samples.

Once the detection window has been established, further tests will be carried out in India where bacterial meningitis is a major cause of mortality. The study will be conducted at the Manipal Hospital in Bangalore, Karnataka. Manipal Hospital was also selected because it is a referral center with access to the current gold standard modalities for detecting bacterial or HSV infection in CSF. ¹⁴C-FIAU will be purchased from a commercial vendor such as, e.g., Moravek Biochemicals and Radiochemicals, Brea, Calif. Governmental and institutional approval will be obtained prior to initiation of this clinical trial. Clinicians most likely to triage and treat patients with meningitis will be educated on the trial. CSF samples and microbiologic data from patients suspected of having meningitis will be collected in a prospective manner.

In brief, a fraction (about 10%, or about 0.5-5 ml) of the CSF sample obtained from patients suspected of having meningitis will be incubated with ¹⁴C-FIAU and processed in the manner described above. It is expected that patients with true bacterial or HSV meningitis will have microbes in the CSF that will incorporate r-FIAU and increase the radioactivity within the sample. If the patient does not have bacterial or HSV meningitis, then the counts within the CSF will be within a ‘normal’ range. The goal is to define a threshold radioactivity level above which the CSF is defined as ‘positive’. If the CSF has ‘positive’ levels of ¹⁴C-FIAU, the patient may have bacterial or HSV meningitis and requires antimicrobial therapy. If r-FIAU can stratify patients appropriately as expected, clinicians will be able to rapidly determine whether antibiotics are indicated.

This stratification can potentially save substantial cost and decrease inappropriate antibiotic use that contributes to microbial resistance. Furthermore, prompt and appropriate use of antibiotics will lead to timely recovery and reduce the morbidity and mortality associated with meningitis. A sensitivity and specificity of greater than 90% is targeted for this assay. If successful, the next steps would be to perform large scale randomized prospective trials where CSF samples will be either analyzed with the methods of the present invention or with existing diagnostics. This will confirm that the present invention will improve outcomes and decrease costs when compared to current methods of detection.

Epidemiological studies will be performed to analyze the outcomes of patients who were diagnosed with bacterial or HSV meningitis using this method as compared to the current standards of practice. Detailed records will be maintained regarding the time required to make the diagnosis using this assay compared to the conventional studies. Medical and microbiological records will be analyzed to study the time required to initiate anti-microbial therapy based on this assay versus current technology. The false positive and negative rates will be calculated for this method. Based on those results, the number of patients who would have had inappropriate antimicrobial regimens had this method been used exclusively may be extrapolated. After several months of implementation of this method, antibiotic sensitivity will be tested in microbial isolates from CSF to determine if this method alters the patterns of resistance.

### Example 8

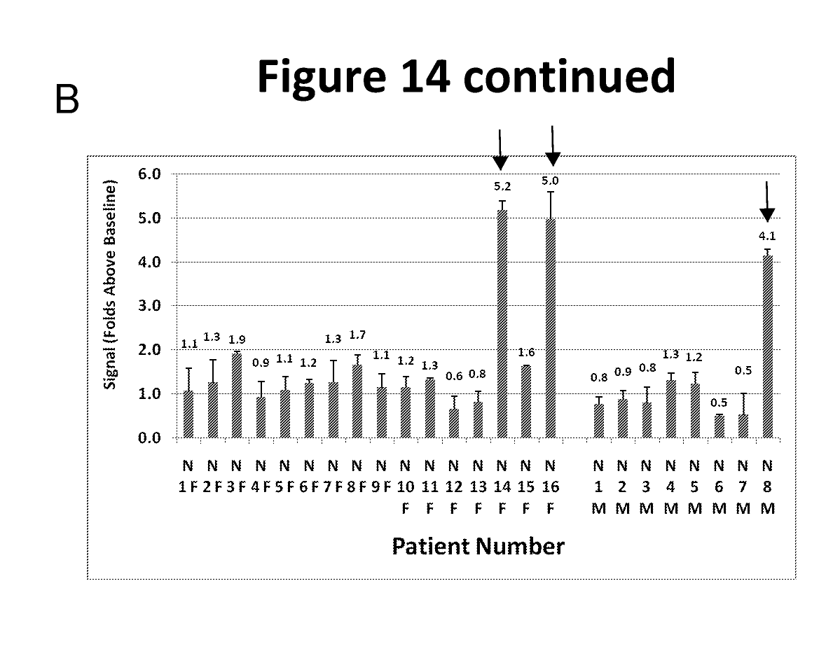

1 ml of a human donor urine sample was added to 1 ml of SOB media containing 0.88 μM ¹⁴C FIAU with a specific activity of 200 mCi/mmol. The mixture was incubated for 6 hours at 37° C. in an agitation incubator. After the incubation period, the mixture was passed through a 0.22 μm Millipore nitrocellulose filter. The filter was then washed with 40 ml of 1×PBS using a syringe. The CPM was determined using a scintillation counter. The data was normalized with respect to the background level obtained on a filter without cells. Values less than 2× above background were considered as negative. All samples were separately tested with urinary tract infection (UTI) urinary test strips, and subsequently cultured on an agar plate to confirm the presence of bacteria. The results are shown in FIG. 14 and in Table 2 below. Although the testing of the donor samples were evaluated in a blinded fashion, the data in Table 2 and FIG. 14 were sorted for the sake of convenience. In the Table below, the donor samples are grouped by UTI positive (P) and negative (N) samples according to the dip stick test. The samples are further subdivided into male (M) and female (F). The ¹⁴C-FIAU test has a 35.29% false positive rate and a 21.43% false negative rate as compared to the urinary dip stick test.

TABLE 2
Comparison of ¹⁴C-FIAU test against
Dip Stick and Agar Plate Culture.

| Donor ID | ¹⁴C-FIAU | Dip Stick | Agar Plate |
|---|---|---|---|
| P 1 F | P | P | P |
| P 2 F | N | P | N |
| P 3 F | P | P | P |
| P 4 F | P | P | P |
| P 5 F | P | P | P |
| P 6 F | P | P | N |
| P 7 F | N | P | N |
| P 8 F | N | P | N |
| P 9 F | N | P | N |
| P 10 F | P | P | N |
| P 1 M | P | P | P |
| P 2 M | P | P | P |
| P 3 M | N | P | P |
| P 4 M | P | P | N |
| P 5 M | N | P | P |
| P 6 M | P | P | P |
| P 7 M | P | P | P |
| N 1 F | N | N | N |
| N 2 F | N | N | N |
| N 3 F | N | N | P |
| N 4 F | N | N | N |
| N 5 F | N | N | N |
| N 6 F | N | N | P |
| N 7 F | N | N | N |
| N 8 F | N | N | N |
| N 9 F | N | N | N |
| N 10 F | N | N | N |
| N 11 F | N | N | N |
| N 12 F | N | N | P |
| N 13 F | N | N | N |
| N 14 F | P | N | N |
| N 15 F | N | N | N |
| N 16 F | P | N | N |
| N 1 M | N | N | N |
| N 2 M | N | N | N |
| N 3 M | N | N | N |
| N 4 M | N | N | N |
| N 5 M | N | N | N |
| N 6 M | N | N | N |
| N 7 M | N | N | N |
| N 8 M | P | N | N |

### Example 9

Similar methods may be performed on other sites of suspected infection, such as testing joint fluid in patients with prosthesis infection. Briefly, the normal range of incorporation of r-FIAU (or another nucleoside analog) by microbes in bodily fluids, such as joint fluid, will be first established. Then the incorporation of r-FIAU by microbes in bodily fluids of patients having an infection will be determined. After broad implementation of this invention, the true sensitivity, specificity, positive and negative predictive values will be calculated.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for detecting whether a subject has an infection comprising:
(a) incubating a test sample from a subject suspected of having an infection with a labeled molecule that is preferentially incorporated into a pathogenic microorganism for a period of time sufficient for the pathogenic microorganism to incorporate the labeled molecule;
(b) removing any unincorporated labeled molecule from the test sample; and
(c) detecting the labeled molecule within the pathogenic microorganism, if any, in the test sample, wherein the presence of labeled molecule within the pathogenic microorganism indicates that the subject has an infection.

2. The method according to claim 1, wherein the infection is a bacterial infection or a viral infection.

3. The method according to claim 2, wherein the infection is selected from the group consisting of bacterial meningitis, HSV meningitis, bacterial pneumonia, urinary tract infection, an infection associated with a prosthetic, abscess, bacterial blood infection, bacterial periotonitis, bacterial pulmonary effusion, skin infection, soft tissue infection, post-surgical infection, and an infection related to implanted, non-prosthetic, hardware.

4. The method according to claim 1, wherein the test sample is a body fluid or a body tissue.

5. The method according to claim 4, wherein the body fluid is selected from the group consisting of whole blood, serum, plasma, interstitial fluid, saliva, ocular lens fluid, cerebro-spinal fluid, sweat, urine, stool, milk, ascites fluid, mucous, nasal fluid, sputum, synovial fluid, peritoneal fluid, vaginal fluid, menses, amniotic fluid, semen, gastric juice, vomit, lymph, and post-operative fluid collections.

6. The method according to claim 5, wherein the body fluid is cerebro-spinal fluid.

7. The method according to claim 5, wherein the body fluid is sputum.

8. The method according to claim 5, wherein the body tissue is a biopsy or a scraping.

9. The method according to claim 8, wherein the biopsy is a bone or organ biopsy.

10. The method according to claim 8, wherein the scraping is a skin or mucosal scraping.

11. The method according to claim 1, wherein the labeled molecule is selected from the group consisting of a labeled nucleoside analog, a labeled non-nucleoside analog, and mixtures thereof.

12. The method according to claim 11, wherein the labeled nucleoside analog is selected from the group consisting of 2′-fluoro-5-iodo-1-β-D-arabinofuranosyluracil (FIAU), 2′-fluoro-5-methyl-1-β-D-arabinofuranosyluracil (FMAU), 2′-fluoro-5-fluoro-1-β-D-arabinofuranosyuracil (FFAU), 2′-fluoro-5-iodo-1-β-D-arabinofuranosyl-cytosine (FIAC), 2′-fluoro-1-β-D-arabinofuranosyluracil (FAU), 1-(2′deoxy-2′-fluoro-β-D-arabinofuranosyl)-5-ethyluracil (FEAU), 3′-deoxy-3′-fluorothymidine (FLT), 9-[4′-fluoro-3′-(hydroxymethyl)butyl]guanine (FHBG), 9-[(3′-fluoro-1′-hydroxy-2′-propoxy)methyl]guanine (FHPG), 2-amino-9-[4-hydroxy-3-(hydroxymethyl)butyl]-6,9-dihydro-3H-purin-6-one (penciclovir), 2-amino-9-((2-hydroxyethoxy)methyl)-1H-purin-6(9H)-one (acyclovir), 2-amino-9-{[(1,3-dihydroxypropan-2-yl)oxy]methyl}-6,9-dihydro-3H-purin-6-one (ganciclovir), and combinations thereof.

13. The method according to claim 11, wherein the labeled non-nucleoside analog is an antibiotic selected from the group consisting of an aminoglycoside, an ansamycin, a carbacephem, a carbapenem, a cephalosporin, a glycopeptide, a lincosamide, a lipopeptide, a macrolide, a monobactam, a nitrofuran, a penicillin, a penicillin combination drug, a polypeptide, a quinolone, a sulphonamide, a polycyclic naphthacene carboxamide derivative, an anti-mycobacteria drug, an unclassified conventional antibacterial drug, and combinations thereof.

14. The method according to claim 13, wherein the antibiotic is selected from the group consisting of Amikacin, Gentamicin, Kanamycin, Neomycin, Netilmicin, Tobramycin, Paromomycin, Geldanamycin, Herbimycin, Loracarbef, Thienamycin, Ertapenem, Doripenem, Imipenem/Cilastatin, Meropenem, Cefadroxil, Cefazolin, Cefalothin, Cefalexin, Cefaclor, Cefamandole, Cefoxitin, Cefprozil, Cefuroxime, Cefixime, Cefmetazole, Cefdinir, Cefditoren, Cefoperazone, Cefotaxime, Cefpodoxime, Ceftazidime, Ceftibuten, Ceftizoxime, Ceftriaxone, Cefepime, Cefetamet, Cefonicid, Cefotetan, Cefsulodin, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spectinomycin, Aztreonam, Furazolidone, Nitrofurantoin, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Cinoxacin, Fleroxacin, Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, Trimethoprim-Sulfamethoxazole (Co-trimoxazole) (TMP-SMX), Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampin, Rifabutin, Rifapentine, Streptomycin, Arsphenamine, Chloramphenicol, Fosfomycin, Fusidic acid, Linezolid, Metronidazole, Mupirocin, Platensimycin, Quinupristin/Dalfopristin, Rifaximin, Thiamphenicol, Tigecycline, Tinidazole, a pharmaceutically acceptable salts thereof, and combinations thereof.

15. The method according to claim 1, wherein the label is selected from the group consisting of a radioactive label, an enzymatic label, a chemiluminescent label, and a fluorescent label.

16. The method according to claim 15, wherein the labeled nucleoside analog is a radio-labeled FIAU (r-FIAU).

17. The method according to claim 16, wherein the r-FIAU is selected from the group consisting of ¹²⁴I-, ¹²⁵I-, ¹⁸F-, and ¹⁴C-labeled FIAU.

18. The method according to claim 1, wherein the subject is a mammal.

19. The method according to claim 18, wherein the mammal is selected from the group consisting of a human, a laboratory animal, a domestic animal, and an agricultural animal.

20. The method according to claim 19, wherein the subject is a human.

21. A method for detecting whether a subject has an infection comprising:
(a) incubating a test sample from a subject suspected of having an infection with a labeled nucleoside analog that is preferentially phosphorylated by a thymidine kinase from a pathogenic microorganism for a period of time sufficient to phosphorylate the labeled nucleoside analog by the thymidine kinase of the pathogenic microorganism;
(b) removing any unphosphorylated or unsequestered labeled nucleoside analog from the test sample; and
(c) detecting labeled nucleoside analog sequestered in the pathogenic microorganism, if any, in the test sample, wherein the presence of labeled nucleoside analog sequestered in the pathogenic microorganism indicates that the subject has an infection.

22. The method according to claim 21, wherein the infection is a bacterial infection or a viral infection.

23. The method according to claim 22, wherein the infection is selected from the group consisting of bacterial meningitis, HSV meningitis, bacterial pneumonia, urinary tract infection, an infection associated with a prosthetic, abscess, bacterial blood infection, bacterial periotonitis, bacterial pulmonary effusion, skin infection, soft tissue infection, post-surgical infection, and an infection related to implanted, non-prosthetic, hardware.

24. The method according to claim 21, wherein the test sample is a body fluid or a body tissue.

25. The method according to claim 24, wherein the body fluid is selected from the group consisting of whole blood, serum, plasma, interstitial fluid, saliva, ocular lens fluid, cerebro-spinal fluid, sweat, urine, stool, milk, ascites fluid, mucous, nasal fluid, sputum, synovial fluid, peritoneal fluid, vaginal fluid, menses, amniotic fluid, semen, gastric juice, vomit, lymph, and post-operative fluid collections.

26. The method according to claim 25, wherein the body fluid is cerebro-spinal fluid.

27. The method according to claim 25, wherein the body fluid is sputum.

28. The method according to claim 24, wherein the body tissue is a biopsy or a scraping.

29. The method according to claim 28, wherein the biopsy is a bone or organ biopsy.

30. The method according to claim 28, wherein the scraping is selected from skin or mucosal scrapings.

31. The method according to claim 21, wherein the nucleoside analog is selected from the group consisting of 2′-fluoro-5-iodo-1-β-D-arabinofuranosyluracil (FIAU), 2′-fluoro-5-methyl-1-β-D-arabinofuranosyluracil (FMAU), 2′-fluoro-5-fluoro-1-β-D-arabinofuranosyuracil (FFAU), 2′-fluoro-5-iodo-1-β-D-arabinofuranosyl-cytosine (FIAC), 2′-fluoro-1-β-D-arabinofuranosyluracil (FAU), 1-(2′deoxy-2′-fluoro-β-D-arabinofuranosyl)-5-ethyluracil (FEAU), 3′-deoxy-3′-fluorothymidine (FLT), 9-[4′-fluoro-3′-(hydroxymethyl)butyl]guanine (FHBG), 9-[(3′-fluoro-1′-hydroxy-2′-propoxy)methyl]guanine (FHPG), 2-amino-9-[4-hydroxy-3-(hydroxymethyl)butyl]-6,9-dihydro-3H-purin-6-one (penciclovir), 2-amino-9-((2-hydroxyethoxy)methyl)-1H-purin-6(9H)-one (acyclovir), 2-amino-9-{[(1,3-dihydroxypropan-2-yl)oxy]methyl}-6,9-dihydro-3H-purin-6-one (ganciclovir), and combinations thereof.

32. The method according to claim 21, wherein the label is selected from the group consisting of a radioactive label, an enzymatic label, a chemiluminescent label, and a fluorescent label.

33. The method according to claim 32, wherein the labeled nucleoside analog is a radio-labeled FIAU (r-FIAU).

34. The method according to claim 33, wherein the r-FIAU is selected from the group consisting of ¹²⁴I-, ¹²⁵I-, ¹⁸F-, and ¹⁴C-labeled FIAU.

35. The method according to claim 21, wherein the subject is a mammal.

36. The method according to claim 35, wherein the mammal is selected from the group consisting of a human, a laboratory animal, a domestic animal, and an agricultural animal.

37. The method according to claim 36, wherein the subject is a human.

38. A method for detecting the presence of a pathogenic microorganism in a test sample comprising:
(a) contacting the test sample with a labeled nucleoside analog that is preferentially phosphorylated by a thymidine kinase of the pathogenic microorganism, if any, in the sample; and
(b) detecting whether the labeled nucleoside analog has been phosphorylated or incorporated into the pathogenic microorganism.

39. The method according to claim 38, wherein the test sample is obtained from a food source.

40. The method according to claim 39, wherein the test sample is obtained from a body fluid or a body tissue.

41. A kit for detecting the presence of a pathogenic microorganism in a test sample, the kit comprising:
(a) a labeled nucleoside analog that is preferentially phosphorylated by a thymidine kinase of a pathogenic microorganism;
(b) a container for contacting the labeled nucleoside analog with a test sample suspected of containing the pathogenic microorganism; and
(c) a device for capturing the pathogenic microorganism, if any, after contacting the labeled nucleoside analog with the sample in the container.

42. The kit according to claim 41 further comprising a first reagent for washing the device to remove excess and unsequestered labeled nucleoside analog.

43. The kit according to claim 41 further comprising a second reagent for use in the container for suspending the labeled nucleoside analog with the test sample suspected of containing a pathogenic microorganism.

44. A method for determining whether a subject is a candidate for antibiotic treatment comprising:
(a) incubating a test sample from a subject suspected of having an infection with a labeled nucleoside analog that is preferentially phosphorylated by a thymidine kinase from a pathogenic microorganism;
(b) removing any unphosphorylated or unsequestered labeled nucleoside analog from the test sample; and
(c) detecting, in the test sample, labeled nucleoside analog sequestered in the pathogenic microorganism, if any, wherein the presence of labeled nucleoside analog sequestered in the pathogenic microorganism indicates that the subject has an infection and is a candidate for antibiotic treatment.

45. A method for monitoring the efficacy of an antibiotic treatment in a subject in need thereof comprising:
(a) incubating a test sample from a subject receiving antibiotic treatment with a labeled nucleoside analog that is preferentially phosphorylated by a thymidine kinase of a pathogenic microorganism;
(b) removing any unphosphorylated or unsequestered labeled nucleoside analog from the test sample; and
(c) detecting, in the test sample, labeled nucleoside analog sequestered in the pathogenic microorganism, if any, wherein the presence and intensity, if any, of incorporated labeled nucleoside analog sequestered in the pathogenic microorganism provides an indication of the efficacy of the antibiotic treatment.

46. A method for detecting bacterial or herpes simplex virus (HSV) meningitis in a subject suspected of having such a disease comprising:
(a) incubating a sample of cerebro-spinal fluid from the subject with ¹⁴C-labeled FIAU that is preferentially phosphorylated by a thymidine kinase of a pathogenic microorganism;
(b) removing any unphosphorylated or unsequestered ¹⁴C-labeled FIAU from the sample of cerebro-spinal fluid; and
(c) detecting, in the cerebro-spinal fluid, ¹⁴C-labeled FIAU sequestered in the pathogenic microorganism, if any, wherein the presence of incorporated ¹⁴C-labeled FIAU sequestered in the pathogenic microorganism indicates that the subject has bacterial or HSV meningitis.

47. A kit for detecting bacterial or HSV meningitis in a subject suspected of having such a disease, the kit comprising:
(a) ¹⁴C-labeled FIAU;
(b) a container for contacting the ¹⁴C-labeled FIAU with a sample of cerebro-spinal fluid from the subject; and
(c) a device for capturing a pathogenic microorganism containing ¹⁴C-labeled FIAU from the sample, if any.

48. A method for detecting bacterial pneumonia in a subject suspected of having such a disease comprising:
(a) incubating a sputum sample from the subject with ¹⁴C-labeled FIAU that is preferentially phosphorylated by a thymidine kinase of a pathogenic microorganism;
(b) removing any unphosphorylated or unsequestered ¹⁴C-labeled FIAU from the sputum sample; and
(c) detecting, in the sputum sample, ¹⁴C-labeled FIAU sequestered in the pathogenic microorganism, if any, wherein the presence of ¹⁴C-labeled FIAU sequestered in the pathogenic microorganism indicates that the subject has bacterial pneumonia.

49. A kit for detecting bacterial pneumonia in a subject suspected of having such a disease, the kit comprising:
(a) ¹⁴C-labeled FIAU;
(b) a container for contacting the ¹⁴C-labeled FIAU with a sputum sample from the subject; and
(c) a device for capturing a pathogenic microorganism containing ¹⁴C-labeled FIAU from the sample, if any.

50. A method for detecting whether a subject has a bacterial infection and, if so, for determining whether an antibiotic has an effect on a pathogenic microorganism causing the bacterial infection, comprising:
(a) incubating a test sample from a subject suspected of having a bacterial infection with a labeled nucleoside analog that is preferentially incorporated into a pathogenic microorganism in the presence and in the absence of an antibiotic for a period of time sufficient for the pathogenic microorganism to incorporate the labeled nucleoside analog;
(b) removing any unincorporated labeled nucleoside analog from the test sample;
(c) detecting the labeled nucleoside analog within the pathogenic microorganism, if any, in the test sample, wherein the presence of labeled nucleoside analog within the pathogenic microorganism indicates that the subject has an infection; and
(d) comparing the amount of labeled nucleoside analog in the pathogenic microorganism incubated in the presence and in the absence of the antibiotic, wherein a reduction in the amount of labeled nucleoside analog in the pathogenic microorganism in the presence of the antibiotic indicates that the antibiotic has an effect on the pathogenic microorganism.

51. A method for detecting whether a subject has a bacterial infection and, if so, for selecting a treatment for the infection, comprising:
(a) incubating a test sample from a subject suspected of having a bacterial infection with a labeled nucleoside analog that is preferentially incorporated into a pathogenic microorganism in the presence and in the absence of an antibiotic for a period of time sufficient for the pathogenic microorganism to incorporate the labeled nucleoside analog;
(b) removing any unincorporated labeled nucleoside analog from the test sample;
(c) detecting the labeled nucleoside analog within the pathogenic microorganism, if any, in the test sample, wherein the presence of labeled nucleoside analog within the pathogenic microorganism indicates that the subject has an infection; and
(d) comparing the amount of labeled nucleoside analog in the pathogenic microorganism incubated in the presence and in the absence of the antibiotic, wherein a reduction in the amount of labeled nucleoside analog in the pathogenic microorganism in the presence of the antibiotic indicates that the antibiotic is a candidate drug for treating the bacterial infection.

52. The method according to claim 50 or 51, wherein the antibiotic in the incubation step comprises a panel of antibiotics.

53. The method according to claim 50 or 51, further comprising identifying the antibiotic corresponding to the highest reduction in the amount of labeled nucleoside analog in the pathogenic microorganism.

54. The method according to claim 50 or 51, wherein the antibiotic is selected from the group consisting of an aminoglycoside, an ansamycin, a carbacephem, a carbapenem, a cephalosporin, a glycopeptide, a lincosamide, a lipopeptide, a macrolide, a monobactam, a nitrofuran, a penicillin, a penicillin combination drug, a polypeptide, a quinolone, a sulphonamide, a polycyclic naphthacene carboxamide derivative, an anti-mycobacteria drug, an unclassified conventional antibacterial drug, and combinations thereof.

55. The method according to claim 54, wherein the aminoglycoside is selected from the group consisting of Amikacin, Gentamicin, Kanamycin, Neomycin, Netilmicin, Tobramycin, Paromomycin, pharmaceutically acceptable salts thereof, and combinations thereof.

56. The method according to claim 54, wherein the ansamycin is selected from the group consisting of geldanamycin, herbimycin, pharmaceutically acceptable salts thereof, and combinations thereof.

57. The method according to claim 54, wherein the carbacephem is selected from the group consisting of loracarbef pharmaceutically acceptable salts thereof, and combinations thereof.

58. The method according to claim 54, wherein the carbapenem is selected from the group consisting of Thienamycin, Ertapenem, Doripenem, Imipenem/Cilastatin, Meropenem, pharmaceutically acceptable salts thereof, and combinations thereof.

59. The method according to claim 54, wherein the cephalosporin is selected from the group consisting of Cefadroxil, Cefazolin, Cefalothin, Cefalexin, Cefaclor, Cefamandole, Cefoxitin, Cefprozil, Cefuroxime, Cefixime, Cefmetazole, Cefdinir, Cefditoren, Cefoperazone, Cefotaxime, Cefpodoxime, Ceftazidime, Ceftibuten, Ceftizoxime, Ceftriaxone, Cefepime, Cefetamet, Cefonicid, Cefotetan, Cefsulodin, Ceftobiprole, pharmaceutically acceptable salts thereof, and combinations thereof.

60. The method according to claim 54, wherein the glycopeptide is selected from the group consisting of Teicoplanin, Vancomycin, Telavancin, pharmaceutically acceptable salts thereof, and combinations thereof.

61. The method according to claim 54, wherein the lincosamide is selected from the group consisting of Clindamycin, Lincomycin, pharmaceutically acceptable salts thereof, and combinations thereof.

62. The method according to claim 54, wherein the lipopeptide is selected from the group consisting of daptomycin, pharmaceutically acceptable salts thereof, and combinations thereof.

63. The method according to claim 54, wherein the macrolide is selected from the group consisting of Azithromycin, Clarithromycin, Dirithromycin, Erythromycin, Roxithromycin, Troleandomycin, Telithromycin, Spectinomycin, pharmaceutically acceptable salts thereof, and combinations thereof.

64. The method according to claim 54, wherein the monobactam is selected from the group consisting of aztreoman, pharmaceutically acceptable salts thereof, and combinations thereof.

65. The method according to claim 54, wherein the nitrofuran is selected from the group consisting of furazolidone, nitrofurantoin, pharmaceutically acceptable salts thereof, and combinations thereof.

66. The method according to claim 54, wherein the penicillin is selected from the group consisting of Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, pharmaceutically acceptable salts thereof, and combinations thereof.

67. The method according to claim 54, wherein the penicillin combination drug is selected from the group consisting of Amoxicilin/clavulanate, Ampicillin/sulbactam, Piperacillin/tazobactam, Ticarcillin/clavulanate, pharmaceutically acceptable salts thereof, and combinations thereof.

68. The method according to claim 54, wherein the polypeptide is selected from the group consisting of Bacitracin, Colistin, Polymyxin B, pharmaceutically acceptable salts thereof, and combinations thereof.

69. The method according to claim 54, wherein the quinolone is selected from the group consisting of Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Cinoxacin, Fleroxacin, pharmaceutically acceptable salts thereof, and combinations thereof.

70. The method according to claim 54, wherein the sulfonamide is selected from the group consisting of Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, Trimethoprim-Sulfamethoxazole (Co-trimoxazole) (TMP-SMX), pharmaceutically acceptable salts thereof, and combinations thereof.

71. The method according to claim 54, wherein the polycyclic naphthacene carboxamide derivative is selected from the group consisting of Demeclocycline, Doxycycline, Minocycline, Oxytetracycline, Tetracycline, pharmaceutically acceptable salts thereof, and combinations thereof.

72. The method according to claim 54, wherein the anti-mycobacteria drug is selected from the group consisting of Clofazimine, Dapsone, Capreomycin, Cycloserine, Ethambutol, Ethionamide, Isoniazid, Pyrazinamide, Rifampin, Rifabutin, Rifapentine, Streptomycin, pharmaceutically acceptable salts thereof, and combinations thereof.

73. The method according to claim 54, wherein the unclassified conventional antibacterial drug is selected from the group consisting of Arsphenamine, Chloramphenicol, Fosfomycin, Fusidic acid, Linezolid, Metronidazole, Mupirocin, Platensimycin, Quinupristin/Dalfopristin, Rifaximin, Thiamphenicol, Tigecycline, Tinidazole, pharmaceutically acceptable salts thereof, and combinations thereof.

74. The method according to claim 50 or 51, wherein the antibiotic is selected from the group consisting of Amikacin, Gentamicin, Kanamycin, Neomycin, Netilmicin, Tobramycin, Paromomycin, Geldanamycin, Herbimycin, Loracarbef, Thienamycin, Ertapenem, Doripenem, Imipenem/Cilastatin, Meropenem, Cefadroxil, Cefazolin, Cefalothin, Cefalexin, Cefaclor, Cefamandole, Cefoxitin, Cefprozil, Cefuroxime, Cefixime, Cefmetazole, Cefdinir, Cefditoren, Cefoperazone, Cefotaxime, Cefpodoxime, Ceftazidime, Ceftibuten, Ceftizoxime, Ceftriaxone, Cefepime, Cefetamet, Cefonicid, Cefotetan, Cefsulodin, Ceftobiprole, Teicoplanin, Vancomycin, Telavancin, Clindamycin, Lincomycin, Daptomycin, Azithromycin, Clarithromycin, Dirithromycin, Erythromycin, Roxithromycin, Troleandomycin, Telithromycin, Spectinomycin, Aztreonam, Furazolidone, Nitrofurantoin, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Cinoxacin, Fleroxacin, Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, Trimethoprim-Sulfamethoxazole (Co-trimoxazole) (TMP-SMX), Demeclocycline, Doxycycline, Minocycline, Oxytetracycline, Tetracycline, Clofazimine, Dapsone, Capreomycin, Cycloserine, Ethambutol, Ethionamide, Isoniazid, Pyrazinamide, Rifampin, Rifabutin, Rifapentine, Streptomycin, Arsphenamine, Chloramphenicol, Fosfomycin, Fusidic acid, Linezolid, Metronidazole, Mupirocin, Platensimycin, Quinupristin/Dalfopristin, Rifaximin, Thiamphenicol, Tigecycline, Tinidazole, pharmaceutically acceptable salts thereof, and combinations thereof.

75. The method according to claim 50 or 51, wherein the labeled nucleoside analog is selected from the group consisting of 2′-fluoro-5-iodo-1-β-D-arabinofuranosyluracil (FIAU), 2′-fluoro-5-methyl-1-β-D-arabinofuranosyluracil (FMAU), 2′-fluoro-5-fluoro-1-β-D-arabinofuranosyuracil (FFAU), 2′-fluoro-5-iodo-1-β-D-arabinofuranosyl-cytosine (FIAC), 2′-fluoro-1-β-D-arabinofuranosyluracil (FAU), 1-(2′deoxy-2′-fluoro-β-D-arabinofuranosyl)-5-ethyluracil (FEAU), 3′-deoxy-3′-fluorothymidine (FLT), 9-[4′-fluoro-3′-(hydroxymethyl)butyl]guanine (FHBG), 9-[(3′-fluoro-1′-hydroxy-2′-propoxy)methyl]guanine (FHPG), 2-amino-9-[4-hydroxy-3-(hydroxymethyl)butyl]-6,9-dihydro-3H-purin-6-one (penciclovir), 2-amino-9-((2-hydroxyethoxy)methyl)-1H-purin-6(9H)-one (acyclovir), 2-amino-9-{[(1,3-dihydroxypropan-2-yl)oxy]methyl}-6,9-dihydro-3H-purin-6-one (ganciclovir), and combinations thereof.

76. A method for detecting the presence of a pathogenic bacteria in a test sample and, if such a pathogenic bacteria is present in the test sample, for identifying a candidate antibiotic for treating or ameliorating the effects of the pathogenic bacteria, comprising:
(a) contacting the test sample with a labeled nucleoside analog that is preferentially phosphorylated or incorporated by a thymidine kinase of the pathogenic bacteria;
(b) further contacting the test sample of step (a) with a panel of antibiotics including one or more controls;
(c) detecting whether the labeled nucleoside analog has been phosphorylated or incorporated into the pathogenic bacteria; and
(d) comparing the amount of phosphorylated or incorporated labeled nucleoside analog in the pathogenic bacteria exposed to each antibiotic or control in the panel and identifying the antibiotic from the panel, which corresponds to the largest reduction in the amount of phosphorylated or incorporated labeled nucleoside analog in the pathogenic bacteria, wherein the antibiotic identified in step (d) indicates the nature of the bacteria causing the infection and provides an antibiotic selectivity profile to a medical professional.

77. A kit for detecting the presence of a pathogenic bacteria in a test sample and for determining whether an antibiotic has an effect on the pathogenic bacteria comprising:
(a) a labeled nucleoside analog that is preferentially phosphorylated by a thymidine kinase of a pathogenic bacteria;
(b) a panel of antibiotics, including a control;
(c) a container for contacting the labeled nucleoside analog with a test sample suspected of containing the pathogenic bacteria and the panel of antibiotics, including a control; and
(d) a device for capturing the pathogenic bacteria, if any, after contacting the labeled nucleoside analog with the sample in the container.
